(19) United States
(12) Patent Application Publication
(10) Pub. No.: US 2026/0224661 A1
(43) Pub. Date: Aug. 6, 2026

(54) ENHANCEMENT OF SYNAPTOGENESIS BY ACTIVATION OF TENEURINS

(71) Applicant: The Board of Trustees of the Leland Stanford Junior University, Stanford, CA (US)

(72) Inventors: Thomas C. Sudhof, Menlo Park, CA (US); Kathlyn J. Gan, Stanford, CA (US); Xuchen Zhang, Redwood City, CA (US)

(21) Appl. No.: 19/151,957

(22) PCT Filed: Feb. 1, 2024
(86) PCT No.: PCT/US2024/014098

§ 371 Date: Jul. 29, 2025

Related U.S. Application Data
(60) Provisional application No. 63/442,940, filed on Feb. 2, 2023.

(51) Int. Cl. A61K 38/17; G01N 33/68

(52) U.S. Cl. CPC A61K 38/1709; G01N 33/68; G01N 2500/20

(57) ABSTRACT

Methods are provided for enhancing synaptogenesis. It is shown herein that the receptor for the circulating factor SPARCL1 (secreted protein acidic and rich in cysteine-like protein) are teneurin proteins (Tenm1-4). The binding site for SPARCL1 is localized to domain 3, which specifically binds to the C terminal domain of SPARCL1 (SPARCL1-C). Contacting neuronal cells expressing a teneurin protein with SPARC is sufficient to increase synapse formation on the neuronal cells.

### INCORPORATION BY REFERENCE OF SEQUENCE LISTING PROVIDED AS A SEQUENCE LISTING XML FILE

A Sequence Listing is provided herewith as a Sequence Listing XML, (STAN-2070WO_sequence listing), created on Feb. 1, 2024, and having a size of 24,429 bytes. The contents of the Sequence Listing XML are incorporated herein by reference in their entirety.

### BACKGROUND

Neurons communicate by two basic mechanisms: fast, point-to-point information transfer mediated by synaptic transmission, and slower, more widespread signaling mediated by a range of messengers, such as neuropeptides, endocannabinoids, and monoamines. Both mechanisms are used not only to transfer information from one neuron to the next, but also to send signals from brain to other cells in the body.

Synapses are intercellular junctions specialized for fast, point-to-point information transfer from a presynaptic neuron to a postsynaptic cell. At a synapse, a presynaptic terminal secretes neurotransmitters via a canonical release machinery, while a postsynaptic specialization senses neurotransmitters via diverse receptors. Synaptic transmission is effected by presynaptic exocytosis of synaptic vesicles containing neurotransmitters, and detection of these neurotransmitters by postsynaptic receptors. Fast, point-to-point synaptic transmission is enabled by the temporal and spatial restriction of neurotransmitter release and reception and by the precise alignment of pre- and postsynaptic structures at a synaptic junction.

Synapses are diverse, with large differences in properties, such as their neurotransmitter types, release probability, postsynaptic receptor composition, and short- and long-term plasticity. The major activity of synapses is to transfer neurotransmitter signals unidirectionally from the pre to the postsynaptic sides and to computationally change the information encoded in these signals. The properties of a given synapse—the manner by which it computes information—are coordinately regulated by bi-directional signals produced by the pre- and postsynaptic compartments, which shape the computation of synaptically transmitted neurotransmitter signals in a circuit. Thus, synapses are not only minimal computational units in brain that process information during transfer between neurons, but also bi-directional signaling devices that organize the transfer and computation of synaptic information.

Only by establishing synaptic connections can nerve cells organize into networks and acquire information processing capability such as learning and memory. Synapses are progressively reduced in number during normal aging; and are severely disrupted during neurodegenerative diseases. The modulation of synapse formation is of great interest for the treatment of a variety of nervous system disorders. To date, few soluble molecules have been identified that are sufficient to induce or increase the number of CNS synapses. Therefore, finding molecules capable of creating and/or maintaining synaptic connections is an important step in the treatment of neurodegenerative diseases.

### SUMMARY

Methods are provided for enhancing synaptogenesis by contacting a cell expressing a teneurin protein, including for example human Tenm1-4, with at least the C-terminal domain of the circulating synaptogenic factor SPARCL1 (secreted protein acidic and rich in cysteine-like protein). The binding site for SPARCL1 is shown herein to be localized to teneurin domain 3. Non-limiting examples of teneurin domain 3 sequences are provided herein as SEQ ID NO:1-SEQ ID NO:4. A teneurin domain 3 sequence specifically binds to the C terminal domain of SPARCL1 (SPARCL1-C, exemplified herein as the human sequence SEQ ID NO:6, or mouse counterpart SEQ ID NO:7 residues 384-684), or a variant thereof. Contacting neuronal cells expressing a teneurin protein in vivo or in vitro with SPARCL1-C is sufficient to increase synapse formation on the neuronal cells, e.g. to increase glutaminergic synapse formation.

In one embodiment of the invention, methods are provided for screening candidate agents for the ability to enhance synapse formation, by determining whether an agent specifically binds to a Tenm domain 3 sequence, e.g. where the agent is a Tenm agonist. A screening assay may comprise a binding assay to determine specific binding between a polypeptide comprising a Tenm domain 3 sequence, and a candidate agent. Screening may be performed in vitro or in vivo. In an embodiment the binding assay is performed with an isolated Tenm polypeptide, e.g. as a high throughput, in vitro, assay. In an embodiment the binding assay is performed with cells expressing a Tenm protein, where the cells are optionally neural cells. In one embodiment of the invention the neurons are neurons in the central nervous system. In another embodiment, the neurons are peripheral nervous system neurons.

In some embodiments the Tenm protein for screening assays is a human protein. In some embodiments the protein is human Tenm2. In some embodiments the Tenm protein comprises a domain 3 region with at least 75% sequence identity to SEQ ID NO:2, at least 80% sequence identity, at least 85%, at least 90%, at least 95%, at least 99% sequence identity. The domain 3 region sequence may be provided in the context of a full-length Tenm protein, a truncated Tenm protein, etc. The domain 3 sequence may be fused to a convenient carrier protein, e.g. a soluble protein, a protein adhered to a substrate, a membrane-bound protein, etc.

In some embodiments a candidate agent for screening is a polypeptide. In some embodiments a candidate polypeptide is an antibody or antibody-like protein, including, for example single domain binding sequences such as VHH domains, ScFv, etc. In other embodiments, a candidate polypeptide comprises at least a portion of a human SPARCL1 C-terminal domain sequence, e.g. comprising all or a portion of SEQ ID NO:6 or a variant thereof, e.g. a polypeptide with at least 75% sequence identity to SEQ ID NO:6, at least 80% sequence identity, at least 85%, at least 90%, at least 95%, at least 99% sequence identity. A SPARCL1 polypeptide may comprise, for example, all or a portion of the SPARCL1 follistatin domain, all or a portion of the SPARCL1 EF-hand domain, or both such domains. In yet other embodiments, a candidate agent is a small molecule drug.

An initial drug screening assay identifies agents that bind to Tenm domain 3 sequence as as candidate Tenm agonists, where the agent selectively binds to one or more Tenm domain 3 sequences, e.g. domain 3 from Tenm1, Tenm2, Tenm3, Tenm4. Candidate agents may be further validated in a cell-based assay to determine if the candidate Tenm agonist increases synapse formation on Tenm-expressing neuronal cells.

In some embodiments methods are provided for increasing synapse formation on neuronal cells in vivo or in vitro. The method may comprise contacting a neuronal cell expressing at least one Tenm protein with a Tenm agonist as described herein, in an effective dose and period of time to increase synapse formation.

In some embodiments methods are provided for protecting or treating an individual suffering from adverse effects of deficits in synapse function. In some such embodiments a Tenm agonist identified herein is administered to an individual in a dose effective to enhance synaptogenesis and/or synaptic strength. Strengthening of synapses and synaptogenesis are enhanced by contacting neurons with agents that are specific agonists of Tenm protein, e.g. agents that specifically bind to a domain 3 sequence of a Tenm protein, and activate Tenm signaling. A variety of clinical conditions are associated with synapse loss, including dementias such as Alzheimer's disease and Parkinson's disease; traumatic brain injury, epilepsy, and other conditions where synapses fail to form or form inappropriately or become weak and are degraded.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1H: Electrophysiological recordings from human neurons treated with full-length SPARCL1 and SPARCL1 fragments identifies the SPARCL1-C domain as the critical synaptogenic region. A. Domain structures of mouse SPARCL1 constructs B-D. Full-length SPARCL1 and its C-terminal SPARCL1-C domain but not its N-terminal acidic region potently promote synapse formation as monitored by measurements of spontaneous miniature mEPSCs (B, representative traces; C & D, bar graphs of the mEPSC frequency and amplitude, respectively). E-F. Measurements of evoked EPSCs confirm that full-length SPARCL1 and its C-terminal SPARCL1-C domain (labeled as SPARC domain) but not its N-terminal acidic region, enhance synaptic connectivity in cultured human neurons, and that they enhance the NMDAR-mediated responses much more strongly than AMPAR-mediated responses (E, representative traces; F, bar graph of the NMDAR-/AMPAR-EPSC ratio of evoked responses; G & H, bar graphs of the AMPAR-EPSC and NMDAR-EPSC amplitudes, respectively).

FIGS. 2A-2F: Identification of teneurins as SPARCL1 receptors. A. Experimental strategy. B. Silver-stained SDS-gel of the various fractions obtained during the experiment depicted in A (L=mol. wt. standards; lanes 1-8=fractions numbers in A). C. To confirm binding of SPARCL1 to teneurins that were identified as binding in the experiment depicted in A and B, recombinant proteins composed of either the extracellular domains of Tenm2 alone or of SPARCL1 alone or of both mixed together were analyzed by size-exclusion chromatography. Note that SPARCL1 is only detectable in higher-molecular weight fractions (#9-11) when Tenm2 is present, but not when it is absent as shown by the immunoblots on the right. D. Surface binding of SPARCL1 or Cbln1 (a neurexin ligand used as a control) to HEK293 cells expressing Neurexin-1a (Nrxn1a) or Tenm1-4. Note that SPARCL1 binds to all teneurins but not to neurexin-1a whereas Cbln1 exhibits the reverse binding pattern. E & F. Affinity determinations of the interaction of Tenm1 with full-length SPARCL1 (E) or the C-terminal SPARCL1-C domain (F), demonstrating a high-affinity interaction.

FIGS. 3A-3C: Definition of domain 3 of teneurin-2 as the SPARCL1 binding site. A. Domain structure of teneurin-2 (Tenm2). B. Representative images of SPARCL1 binding to HEK293 cells expressing the indicated Tenm2 domain constructs consisting of the listed domains fused to the transmembrane region (FLAG=control). C. Quantification of SPARCL1 binding to the indicated Tenm2 domains.

FIGS. 4A-4G. Demonstration that the follistatin-like domain of SPARCL1 is sufficient for teneurin binding but is unable to boost synapse numbers in cultured neurons. A. Domain structures of additional SPARCL1 constructs. B-E. Surface-binding assays identify specific binding of full-length SPARCL1 (B) and the follistatin-like domain of SPARCL1 (D) to all four teneurins expressed in HEK293 cells, whereas the EF-hand Ca²⁺-binding domain of SPARCL1 does not bind (E), nor does cerebellin-1 as a negative control (C). Cerebellin-1 does, however, bind to Nrxn1α expressed in HEK293 cells, which in turn does not bind to any SPARCL1 fragment. F & G. The follistatin-like domain of SPARCL1 that binds to teneurins is by itself unable to promote synapse formation different from full-length SPARCL1 (F, representative images; G, quantification of synapse density).

### DETAILED DESCRIPTION OF THE EMBODIMENTS

Contact with a Tenm agonist induces new synapse formation in normal neurons and can strengthen existing synapses. The newly-formed synapses may be glutaminergic synapses. The ability to restore synaptogenesis in an adult has important implications for enhancing memory in normal brain; for treatment of Alzheimer's disease (a disease where synapses are lost), as well as promoting new synaptogenesis in repair and regeneration of injured CNS after stroke or spinal cord injury; enhancement of neuromuscular junctions in muscular dystrophy; and the like. Delivery of a Tenm agonist also finds use in combination with administration of neural progenitors or stem cells, or other interventions that increase in neurogenesis, in order to promote functional connections between the nascent neurons and other neurons and effector cells.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases. Chemicals that are known but not commercially available in catalogs may be prepared by custom chemical synthesis houses, where many of the standard chemical supply houses (e.g., those listed above) provide custom synthesis services.

### Definitions

Synapses are asymmetric communication junctions formed between two neurons, or, at the neuromuscular junction (NMJ) between a neuron and a muscle cell. At an archetypal chemical synapse, such as those found at dendritic spines, a mushroom-shaped bud projects from each of two cells and the caps of these buds press flat against one another. At this interface, the membranes of the two cells flank each other across a slender gap, the narrowness of which enables signaling molecules known as neurotransmitters to pass rapidly from one cell to the other by diffusion. This gap, which is about 20 nm wide, is known as the synaptic cleft.

Chemical synapses enable cell-to-cell communication via secretion of neurotransmitters, whereas in electrical synapses signals are transmitted through gap junctions, specialized intercellular channels that permit ionic current flow. In addition to ions, other molecules that modulate synaptic function (such as ATP and second messenger molecules) can diffuse through gap junctional pores. At the mature neuromuscular junction, pre- and postsynaptic membranes are separated by the synaptic cleft containing extracellular proteins that form the basal lamina. Synaptic vesicles are clustered at the presynaptic release site, transmitter receptors are clustered in junctional folds at the postsynaptic membrane, and glial processes surround the nerve terminal.

Synapses are asymmetric both in structure and in how they operate. Only the presynaptic neuron secretes the neurotransmitter, which binds to receptors facing into the synapse from the postsynaptic cell. The pre-synaptic nerve terminal (also called the synaptic button or bouton) generally buds from the tip of an axon, while the post-synaptic target surface typically appears on a dendrite, a cell body, or another part of a cell. The parts of synapses where neurotransmitters are released are called the active zones. At active zones the membranes of the two adjacent cells are held in close contact by cell adhesion proteins. Immediately behind the postsynaptic membrane is an elaborate complex of interlinked proteins called the postsynaptic density. Proteins in the postsynaptic density serve a myriad of roles, from anchoring and trafficking neurotransmitter receptors into the plasma membrane, to anchoring various proteins which modulate the activity of the receptors. The postsynaptic cell need not be a neuron, and can also be gland or muscle cell.

The release of a neurotransmitter is triggered by the arrival of a nerve impulse (or action potential) and occurs through an unusually rapid process of cellular secretion known as exocytosis. Within the pre-synaptic nerve terminal, vesicles containing neurotransmitter are docked at the synaptic membrane. The arriving action potential produces an influx of calcium ions through voltage-dependent, calcium-selective ion channels. Calcium ions then trigger a biochemical cascade which results in vesicles fusing with the presynaptic-membrane and releasing their contents to the synaptic cleft. Vesicle fusion is driven by the action of a set of proteins in the presynaptic terminal known as SNAREs. The membrane added by this fusion is retrieved by endocytosis and recycled for the formation of fresh neurotransmitter-filled vesicles. Receptors on the opposite side of the synaptic gap bind neurotransmitter molecules and respond by opening nearby ion channels in the post-synaptic cell membrane, causing ions to rush in or out and changing the local transmembrane potential of the cell. The resulting change in voltage is called a postsynaptic potential, which can be measured by patch-clamping and other suitable techniques.

Based on the neurotransmitters that they release, chemical synapses are either excitatory or inhibitory. In the central nervous system (CNS), glutamate is the principle excitatory neurotransmitter acting on three types of ionotropic glutamate receptors (AMPA receptors, NMDA receptors and Kainate receptors) to mediate fast excitatory synaptic transmission and induce depolarization in post-synaptic neurons. GABA is the chief inhibitory neurotransmitter in the mature CNS and exerts its action through GABAA-type ionotropic receptors (GABAARs) to achieve fast synaptic inhibition. GABAergic inhibition balances glutamatergic excitatory drive and controls neuronal excitability, integration and output. This synaptic excitation and inhibition balance (E/I balance) is important for proper neural circuit function, and dysregulation of balanced E/l development has been proposed as a potential mechanism for a number of neural development disorders.

SPARCL1 has been associated with modulating the formation of glutamatergic synapses. Glutamatergic synapses are the main excitatory synapses in the brain. These synapses consist of glutamate localized inside presynaptic vesicles and glutamate receptors on the postsynaptic membrane.

During synaptic transmission, nerve impulses are transmitted to the presynaptic terminal, depolarizing the presynaptic membrane, which leads to the influx of calcium ions (Ca2⁺) into the presynapse due to the opening of the voltage-gated calcium channel (VGCC). Glutamate is released into the synaptic cleft through the fusion of synaptic vesicles and presynaptic membranes at the active zone. Released Glu acts on various types of Glu receptors (GluRs) in the postsynaptic membrane, transmitting nerve impulses and exerting multiple physiological roles. Appropriate stimulation of GluRs is necessary for learning and memory. But excessive activation of GluRs in the hippocampus region may produce pathological long-term potentiation (LTP) and may even cause excitotoxicity that will impair cognition and induce neuronal damage & death.

Gamma-aminobutyric acid (GABA) is an amino acid that serves as the primary inhibitory neurotransmitter in the brain and a major inhibitory neurotransmitter in the spinal cord. It exerts its primary function in the synapse between neurons by binding to post-synaptic GABA receptors which modulate ion channels, hyperpolarizing the cell and inhibiting the transmission of an action potential. Disorder in GABA signaling is implicated in a multitude of neurologic and psychiatric conditions. Modulation of GABA signaling is the basis of many pharmacologic treatments in neurology, psychiatry, and anesthesia.

GABA is synthesized in the cytoplasm of the presynaptic neuron from the precursor glutamate by the enzyme glutamate decarboxylase. After synthesis, it is loaded into synaptic vesicles by the vesicular inhibitory amino acid transporter. GABA binds to two major post-synaptic receptors, the GABA-A and GABA-B receptors. The GABA-A receptor is an ionotropic receptor that increases chloride ion conductance into the cell in the presence of GABA. The extracellular concentration of chloride is normally much higher than the intracellular concentration. Consequently, the influx of negatively charged chloride ions hyperpolarizes the cell, inhibiting the creation of an action potential. The GABA-B receptor functions via a metabotropic G-protein coupled receptor which increases postsynaptic potassium conductance and decreases presynaptic calcium conductance, which consequently hyperpolarizes the postsynaptic cell and prevents the conduction of an action potential in the presynaptic cell. Consequently, regardless of binding to GABA-A or GABA-B receptors, GABA serves an inhibitory function.

GABA is involved in complex circuits throughout the central nervous system. For example, GABA is released by striatal neurons in both the direct and indirect pathways projecting to the globus pallidus, which in turn extends GABA neurons to other brain areas, inhibiting unwanted motor signals. Another example is that GABA signaling in the medulla is involved in the maintenance of respiratory rate. Increased GABA signaling reduces the respiratory rate. A third example is found in the spinal cord, where GABA serves in the inhibitory interneurons. These neurons help to integrate excitatory proprioceptive signals, allowing for the spinal cord to integrate sensory information and create smooth movements.

Synaptogenesis. Synaptogenesis, as used herein, refers to the process by which pre- and/or post-synapses form on a neuron. Enhancing synaptogenesis results in an increased number of synapses, while inhibiting synaptogenesis results in a decrease in the number of synapses, or a lack of increase where an increase would otherwise occur. By “augmentation” or “enhancement” of synaptogenesis as used herein, it is meant that the number of functional synapses formed is increased in response to a specified signal. An “activator” or “agonist” is a substance that enhances synaptogenesis.

Synaptogenesis is a dynamic process. During development, more synapses are established than ultimately will be retained. Therefore, the elimination of excess synaptic inputs is a critical step in synaptic circuit maturation. Synapse elimination is a competitive process that involves interactions between pre- and postsynaptic partners. In the CNS, as with the NMJ, a developmental, activity-dependent remodeling of synaptic circuits takes place by a process that may involve the selective stabilization of coactive inputs and the elimination of inputs with uncorrelated activity. The anatomical refinement of synaptic circuits occurs at the level of individual axons and dendrites by a dynamic process that involves rapid elimination of synapses. As axons branch and remodel, synapses form and dismantle with synapse elimination occurring rapidly.

The term “neural stem cell” refers to self-renewing, multipotent cells that firstly generate the radial glial progenitor cells that generate the neurons and glia of the nervous system of all animals during embryonic development. Some neural progenitor stem cells persist in highly restricted regions in the adult vertebrate brain and continue to produce neurons throughout life. NSCs are commonly characterized by a combination of expression of a NSC/astrocyte marker, a NSC/IPC marker, and/or the ciliary protein prominin1 (CD133), as well as their radial morphology and location of the cell body in the stem cell layer of the V-SVZ or the DG.

Stem cells are characterized by their capacity to differentiate into multiple cell types. They undergo symmetric or asymmetric cell division into two daughter cells. In symmetric cell division, both daughter cells are also stem cells. In asymmetric division, a stem cell produces one stem cell and one specialized cell. NSCs primarily differentiate into neurons, astrocytes, and oligodendrocytes.

In the adult mammalian brain, the subgranular zone in the hippocampal dentate gyrus, the subventricular zone around the lateral ventricles, and the hypothalamus (precisely in the dorsal α1, α2 region and the “hypothalamic proliferative region”, located in the adjacent median eminence) have been reported to contain neural stem cells.

NSCs are stimulated to begin differentiation via exogenous cues from the microenvironment, or stem cell niche. Some neural cells are migrated from the SVZ along the rostral migratory stream which contains a marrow-like structure with ependymal cells and astrocytes when stimulated. The ependymal cells and astrocytes form glial tubes used by migrating neuroblasts. The astrocytes in the tubes provide support for the migrating cells as well as insulation from electrical and chemical signals released from surrounding cells. The astrocytes are the primary precursors for rapid cell amplification. The neuroblasts form tight chains and migrate towards the specified site of cell damage to repair or replace neural cells. Neural stem cell proliferation declines as a consequence of aging.

The most widely accepted model of an adult NSC is a radial, glial fibrillary acidic protein-positive cell. Quiescent stem cells are able to remain in the quiescent state due to the renewable tissue provided by the specific niches composed of blood vessels, astrocytes, microglia, ependymal cells, and extracellular matrix present within the brain. Once activated, the develop into active proliferating intermediate cells, which then divide into neuroblasts. The undifferentiated neuroblasts form chains that migrate and develop into mature neurons.

Markers expressed by neural stem cells include Nestin, an intermediate filament protein; Sox2 (Sex-determining region Y (SRY)-Box 2) transcription factor; Musashi-1, an RNA-binding protein; Pax6 (Paired Box 6) transcription factor; notch receptors, CD133, and Olig2.

Teneurins. Teneurins (Tenm) are a family of phylogenetically conserved single-pass transmembrane glycoproteins expressed during pattern formation and morphogenesis. They are expressed by a subset of neurons as well as at sites of pattern formation and morphogenesis. There are four teneurin genes in vertebrates, named teneurin-1 through -4. Teneurins are believed to translocate to the nucleus where they regulate transcriptional activity. Proteins of interest include, without limitation, human Tenm1, human Tenm2, human Tenm3, human Tenm4. In some embodiments, an agent of interest activates human Tenm2 by selective domain 3 binding.

Tenms are composed of an N-terminal cytoplasmic tail, a single transmembrane region (TM), and a large ECR. The ECR of TENs comprises eight epidermal growth factor (EGF) motifs that are followed by the large unknown region composed of domains identified as 2, 3, 4 and 5 (see, for example, Tucker (2018) Teneurins: Domain Architecture, Evolutionary Origins, and Patterns of Expression, Front. Neurosci. 12:938). The TEN2 ECR has an unusual architecture comprising a large cylindrical β-barrel sealed at the bottom by an Immunoglobulin (Ig)-like domain and a β-propeller; and at the top by a C-terminal domain. The teneurin intracellular (IC) domain (~300-400 aa) is located at the N-terminus and contains a number of conserved putative tyrosine phosphorylation sites, two EF-hand-like calcium-binding motifs, and two polyproline domains. These proline-rich stretches are characteristic of SH3-binding sites.

The reference sequences for exemplary human teneurin proteins may be accessed at Genbank, e.g. teneurin-1 isoform 1, accession no. NP_001156750 XP_011529532; teneurin-2 isoform 1, accession no. NP_001116151; teneurin-3 isoform 1, accession no. NP_001402902 XP_047271894; teneurin-4, accession no. NP_001092286 XP_001131144 XP_945556.

Alternatively spliced variants of teneurins may interact with distinct ligands. TENMs are alternatively spliced at two sites within the ECR and include nine- and seven-residue insertions at the EGF repeats and the β-propeller regions, respectively. It has been suggested that an alternatively spliced seven-residue region within the β-propeller acts as a switch to regulate trans-cellular adhesion of TENM2.

As used herein the term Teneurin domain 3 sequence refers to a region of a Tenm protein corresponding to approximately residues 1181-1522 of human Tenm2. Examples of suitable human domain 3 sequences are provided as SEQ ID NO:1-4. A Tenm domain 3 sequence may have at least 75% sequence identity to SEQ ID NO:2, at least 80% sequence identity, at least 85%, at least 90%, at least 95%, at least 99% sequence identity.

SPARCL1. SPARCL1 (also known as Hevin), is a matricellular protein belonging to the SPARC protein family. It is a secreted non-neuronal protein that has been reported to increase synaptogenesis. Human SPARCL1 consists of an N-terminal secretion signal peptide causing an internal follistatin-like domain (FLD), a C-terminal extracellular calcium-binding domain and a highly acidic domain positioned between the signal peptide sequence and the FLD that is 411 amino acids long in SPARCL1 but only 51 amino acids in SPARCL1-C. The human reference protein sequence may be accessed at Genbank, NP_001121782. Reference sequences for the full length human protein are provided as SEQ ID NO:5, the mature mouse protein as SEQ ID NO:7, the SPARC domain as SEQ ID NO:6, and the mouse SPARC domain as SEQ ID NO:7, residues 384-684.

SPARCL1 is reported to selectively increase excitatory, but not inhibitory synapse numbers, enhance excitatory but not inhibitory synaptic transmission, and augment NMDAR-mediated synaptic responses more than AMPAR-mediated synaptic responses. None of these effects are mediated by SPARCL1-binding to neurexins or neuroligins.

The C terminal domain of SPARCL1, referred to herein as SPARCL1-C (or SPARC), is shown herein to bind to and activate teneurin, increasing synapse formation. The follistatin-like domain is sufficient for binding, but both the Follistain-like domain and the EF-hand domain are required for increasing synaptogenesis. A SPARCL1-C sequence may comprise at least a portion of a SPARCL1 sequence, e.g. all or a portion of SEQ ID NO:6 or a variant thereof, or SEQ ID NO:7 residues 384-684 or a variant thereof, e.g. a polypeptide with at least 75% sequence identity, at least 80% sequence identity, at least 85%, at least 90%, at least 95%, at least 99% sequence identity to these reference sequences.

Tenm agonists are agents that selectively bind to and activate Tenm signaling, e.g. thereby increasing synaptogenesis. Binding to a Tenm domain 3 sequence by an agonist of interest may be at least about 10-fold the level of binding to negative control, e.g. neurexin 1a, neuroligin, etc., and may be at least about 100-fold, at least about 1000-fold or more. The binding affinity of a Tenm agonist for a Tenm domain 3 sequence may have a Kd of less than about 500 nM, less than about 100 nM, less than about 50 nM, less than about 1 nM. Molecules of interest as agonists may include proteins, nucleic acids, carbohydrates, antibodies, nanobodies, small molecule drugs, or any other molecules that selectively activate the receptor. The Tenm domain 3 sequence may comprise all or a portion of SEQ ID NO:2. Tenm agonists of interest, or candidate agonists of interest include, without limitation, a SPARCL1 polypeptide or derivative or analog thereof; a polypeptide comprising a Tenm antigen binding domain; and a small molecule drug.

In some embodiments a Tenm agonist, or a candidate agonist for screening purposes, is a polypeptide comprising an antigen binding region (ABR). As used herein, the term ABR refers to a combination of variable heavy (VH and variable light (VL) polypeptides to associate to form a variable region domain. An ABR is the minimum antibody fragment that contains a complete antigen-recognition and binding site. This region consists of heavy- and one light-chain variable domain in tight, non-covalent association, as a single polypeptide or as a dimer. It is in this configuration that the three CDRS of each variable domain interact to define an antigen-binding site on the surface of the domain. Collectively, the six CDRs confer antigen-binding specificity to the antibody. Such an agonist may be in a format selected from, but not limited to, intact IgG, IgE and IgM, bi- or multi-specific antibodies (e.g., Zybodies®, etc), single chain Fvs, Fabs, Small Modular ImmunoPharmaceuticals (“SMIPs™”), single chain or Tandem diabodies (TandAb®), VHHs, Anticalins®, Nanobodies®, minibodies, BiTE® s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, KALBITOR®, etc. as known in the art.

An antibody or ABR or nanobody “that binds” to a Tenm protein domain 3, e.g. SEQ ID NO:2, is one that binds the polypeptide antigen with sufficient affinity that the antibody or binding molecule is useful as a therapeutic agent in activating the receptor, and does not significantly cross-react with other proteins. In such embodiments, the extent of binding of the antibody or other binding molecule to a non-targeted antigen will usually be no more than 10% as determined by fluorescence activated cell sorting (FACS) analysis or radioimmunoprecipitation (RIA).

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VL) domain. The two domains conventionally associate to form a specific binding region, although as well be discussed here, a variety of non-natural configurations of antibodies are known and used in the art.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, VHH, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include 3 CDR sequences, which may be obtained from available antibodies with the desired specificity, or may be obtained from antibodies developed for this purpose. One of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139, each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

In some embodiments, a Tenm agonist is a nanobody. Unless indicated otherwise, the term “immunoglobulin single variable domain” or “ISV” is used as a general term to include but not limited to antigen-binding domains or fragments such as V_{HH} domains or V_{H} or V_{L} domains, respectively. As used herein, a “nanobody” refers to a single-domain antibody, which may be designated sdAb, which is an antibody fragment consisting of a single monomeric variable antibody domain that is able to bind selectively to an antigen. A nanobody may be derived from camelids (V_{H}H fragments) or cartilaginous fishes (V_{NAR} fragments). A nanobody comprises a variable region primarily responsible for antigen recognition and binding and a framework region. The “variable region,” also called the “complementarity determining region” (CDR), comprises loops which differ extensively in size and sequence based on antigen recognition. CDRs are generally responsible for the binding specificity of the nanobody. Distinct from the CDRs is the framework region. The framework region is relatively conserved and assists in overall protein structure. The framework region may comprise a large solvent-exposed surface consisting of a β-sheet and loop structure. A signal sequence, as known in the art, can be included, which is then cleaved from the mature nanobody.

The generation of immunoglobulin single variable domains such as e.g., V_{HH}S or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

The immunoglobulin single variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively.

An amino acid sequence such as e.g. an immunoglobulin single variable domain or polypeptide according to the invention is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195.

The term analog, or derivative, is used herein to refer to a molecule that structurally resembles a molecule of interest but which has been modified or identified in a targeted and controlled manner, by replacing a specific substituent of the reference molecule with an alternate substituent. In some embodiments an analog refers to a polypeptide, such as a SPARC domain, ABD, etc. that has been modified, e.g. by amino acid substitutions, deletions, additions, and other modifications. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry. In some embodiments, an analog of SPARCL1 comprising all or a portion of SEQ ID NO:6 or a variant thereof or SEQ ID NO:7 residues 384-684, as described herein is an agonist of Tenm. In the cases of SPARCL1, some analogs contain a domain comprising at least a portion of SEQ ID NO:6 or SEQ ID NO:7 residues 384-684, e.g. comprising at least 5 contiguous amino acids, at least 10 contiguous amino acids, at least 20 amino acids, at least 30 amino acids, at least 40 amino acids, at least 50 amino, which may be provided as a fusion protein joined to, e.g. a domain that provides for enhanced half-life and stability after administration.

In some embodiments, an analog can be conjugated to additional molecules to provide desired pharmacological properties such as extended half-life. In one embodiment, an analog can be fused to the Fc domain of IgG, albumin, or other molecules to extend its half-life, e.g. by pegylation, glycosylation, and the like as known in the art. In some embodiments the analog is conjugated to a polyethylene glycol molecules or “PEGylated.” The molecular weight of the PEG include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly or via a linker molecule. The processes and chemical reactions necessary to achieve PEGylation of biological compounds is well known in the art.

An analog can be acetylated at the N-terminus, using methods known in the art, e.g. by enzymatic reaction with N-terminal acetyltransferase and, for example, acetyl COA. The analog can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The analogs can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, an analog can comprise polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the analog polypeptide further comprises a C-terminal c-myc epitope tag or other epitope tags, or modifications or fusions to moieties that will enhance blood-brain barrier transport such as transferrin-receptor binding sequences or that will increase the protein's half-life, such as pegylation.

In some embodiments an analog polypeptide is fused to a transporter domain, e.g. penetratin-1; VP-22, TAT, poly-arginine, etc., as known in the art.

For use in the subject methods, any SPARCL1 analog, Tenm agonist, ABD polypeptide, modifications thereof, or a combination of forms may be used. Peptides of interest include fragments of at least about 5 contiguous amino acids, at least about 10 contiguous amino acids, more usually at least about 20 contiguous amino acids, and may comprise 30 or more amino acids, up to a complete domain, or the complete polypeptide.

The sequence of a Tenm agonist, e.g. SPARCL1-C polypeptide, may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Compound Screening

In one embodiment of the invention, methods are provided for screening candidate agents for the ability to enhance synapse formation, by determining whether an agent specifically binds to a Tenm domain 3 sequence, e.g. is a Tenm agonist. A screening assay may comprise a binding assay to determine specific binding between a polypeptide comprising a Tenm domain 3 sequence, and a candidate agent. Screening may be performed in vitro or in vivo. In an embodiment the binding assay is performed with an isolated Tenm polypeptide, e.g. as a high throughput, in vitro assay. In an embodiment the binding assay is performed with cells expressing a Tenm protein, where the cells are optionally neural cells. In one embodiment of the invention the neurons are neurons in the central nervous system. In another embodiment, the neurons are peripheral nervous system neurons.

In some embodiments the Tenm protein for screening assays is a human protein. In some embodiments the protein is human Tenm2. In some embodiments the Tenm protein comprises a domain 3 region with at least 75% sequence identity to SEQ ID NO:2, at least 80% sequence identity, at least 85%, at least 90%, at least 95%, at least 99% sequence identity. The domain 3 region sequence may be provided in the context of a full-length Tenm protein, a truncated Tenm protein, et. The domain 3 sequence may be fused to a convenient carrier protein, e.g. a soluble protein, a protein adhered to a substrate, a membrane-bound protein, etc.

In some embodiments a candidate agent for screening is a polypeptide. In some embodiments a candidate polypeptide is an antibody or antibody-like protein, including, for example single domain binding sequences such as VHH domains, ScFv, etc. In other embodiments, a candidate polypeptide comprises at least a portion of a human SPARCL1 C-terminal domain sequence, e.g. comprising all or a portion of SEQ ID NO:6 or a variant thereof, or SEQ ID NO:7, residues 384-684 or a variant thereof, e.g. a polypeptide with at least 75% sequence identity, at least 80% sequence identity, at least 85%, at least 90%, at least 95%, at least 99% sequence identity to the reference sequence. A SPARCL1 polypeptide may comprise, for example, all or a portion of the SPARCL1 follistatin domain, all or a portion of the SPARCL1 EF-hand domain, or both such domains. In yet other embodiments, a candidate agent is a small molecule drug.

An initial drug screening assay identifies agents that bind to Tenm domain 3 sequence as candidate Tenm agonists, where the agent selectively binds to one or more Tenm domain 3 sequences, e.g. domain 3 from Tenm1, Tenm2, Tenm3, Tenm4. Candidate agents may be further validated in a cell-based assay to determine if the candidate Tenm agonist increases synapse formation on Tenm-expressing neuronal cells.

Candidate agents may be identified by known pharmacology, by structure analysis, by rational drug design using computer based modeling, by binding assays, and the like. In some embodiments a specific Tenm binding molecule is generated, e.g. by modification of SPARCL1 C domain, antibody or nanobody screening, etc. Various in vitro models may be used to determine whether a compound binds to, or otherwise affects Tenm activity. Such candidate compounds may then be used to contact neurons in a culture environment permissive for synaptogenesis. Such compounds may be further tested in an in vivo model for enhanced synaptogenesis.

The term “agent” as used herein describes any molecule, e.g. protein or pharmaceutical, with the capability of modulating synaptogenesis, particularly through a Tenm signaling pathway. Candidate agents encompass numerous chemical classes, including polypeptide. In some embodiments candidate agents are organic molecules, for example small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of candidate binding agents can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

A large number of high-throughout binding/interaction methods are known in the art and useful for determining protein interactions, including without limitation the yeast two-hybrid system (YTH), co-immunoprecipitation (co-IP), fluorescence resonance energy transfer or Förster resonance energy transfer (FRET), tandem affinity purification (TAP), and protein fragment complementation assays (PCAs). Since its invention, the yeast two-hybrid analysis has been widely used to map protein binding partners from diverse cell types. YTH has also contributed to the development of variant technologies for mapping protein interactions in vivo, such as membrane yeast-two hybrid assay (MYTH). MYTH is a split ubiquitin-based two-hybrid analysis, which is designed to overcome limitations of the original YTH system for application to membrane proteins. YTH and the related methods have been widely used for high-throughout studies. FRET refers to an energy transfer process from an excited donor molecule to another nearly acceptor molecule, and is very sensitive to the distance between the donor and acceptor molecules. Recent advances in microscopy and spectroscopy make FRET a powerful technique to monitor spatiotemporal changes in interactions. FRET-based methods are also used for high-throughput screening using protein microarrays.

In PCAs, a reporter (such as a fluorescent protein or an enzyme) is truncated and fused to two proteins of interest. If these two proteins interact with each other, the complementary fragments are capable of proper folding and assembly, reconstituting the reporter activity. One of the PCA methods is bimolecular fluorescence complementation (BiFC), which is based on the reconstitution of a fluorescent protein in vivo. A BiFC allows direct visualization of interactions in living cells. Two proteins of interest are fused to N-terminal or C-terminal non-fluorescent fragment of a fluorescent protein and then expressed in living cells. If an interaction takes place between the two proteins of interest, this will facilitate reconstitution of the fluorescent protein, forming a fluorescent complex. The BiFC signal can then be visualized by fluorescent microscopy or analyzed by flow cytometry without any other special treatment to cells.

Fluorescence resonance energy transfer (FRET) and bioluminescence resonance energy transfer (BRET) are closely related in principle; all involve the nonradioative energy transfer that takes place between two suitable molecules (donor and acceptor) that are in close proximity. The excitation of FRET donor fluorophores needs an extra excitation light, whereas the donor moiety in BRET emits luminescence as a result of an enzymatic reaction. Therefore, BRET sensors do not require external illumination.

CFP-YFP and eCFP-eGFP are typical FRET-based sensor pairs used to study interactions between proteins. In BRET assays, the absence of external illumination removes detector background caused by scattered illumination light and undesired acceptor fluorescence caused by direct excitation, and thus increase the signal/noise ratio. A simple and flexible NanoLuc-based complementation assay (NanoBit, NanoLuc Binary Technology) may overcome the high background noise and low sensitivity issues of FRET and BRET and give access to the profiling of ligands for their ability to induce interaction in high-throughput format.

Several interaction assays based on enzyme fragment complementation, such as PathHunter and NanoBiT, have also been developed to assess interactions. PathHunter uses enzyme fragment complementation of β-galactosidase and its enzyme activity while NanoBit is based on the NanoLuc. In PathHunter assay, β-arrestin is fused to an N-terminal deletion mutant of β-galactosidase that is catalytically inactive, and a protein is tagged at the C-terminus with a small (4 kDa) fragment derived from the deleted N-terminal sequence of β-galactosidase (ProLink). With NanoBit assay, the protein is tagged with SmBit, and the β-arrestin is tagged with LgBit. Both assays are widely accepted in drug-screening laboratories.

Compounds that are initially identified by binding assays can be further tested to validate the activity. The basic format of such methods involves administering a lead compound identified during an initial screen to a cell or animal that serves as a model for humans and then determining the effects on synaptogenesis. Assays may be performed for biochemical indicia of teneurin signaling, e.g. changes in kinase activity, etc., or changes in biological indicia, e.g. synapse formation

For example, synaptogenesis can be quantitated by administering a candidate agent to neurons in culture, and determining the presence of synapses in the absence or presence of the agent. In one embodiment of the invention, the neurons are a primary culture, e.g. of RGCs, mixed cortical and hippocampal neurons; primary cultures of human neurons derived from embryonic stem cells, or other neurons, which may be mixed with glial cells. Purified populations can obtained by conventional methods, such as sequential immunopanning. The cells are cultured in suitable medium, which will usually comprise appropriate growth factors, e.g. CNTF; BDNF; etc. As a positive control, soluble SPARCL1 may be added to certain wells. The neural cells are cultured for a period of time sufficient allow robust process outgrowth and then cultured with a candidate agent for a period of about 1 day to 1 week, to allow synapse formation. For synapse quantification, cells may be stimulated as the EPSC recorded. Alternatively, cultures are fixed, blocked and washed, then stained with antibodies specific synaptic proteins, e.g. synaptotagmin, etc. and visualized with an appropriate reagent, as known in the art. Analysis of the staining may be performed microscopically. In one embodiment, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Number, mean area, mean minimum and maximum pixel intensities, and mean pixel intensities for all synaptotagmin, PSD-95, and colocalized puncta in the image are recorded and saved for analysis.

Further animal models can be utilized in validation studies to determine the effect of a candidate agent on synaptogenesis in vivo, and may include determination of an effect on cognitive function. Specific examples of suitable animals include, but are not limited to, primates, mice, and rats.

### Conditions of Interest for Treatment

By “neurological” or “cognitive” function as used herein, it is meant that the increase of synapses in the brain enhances the patient's ability to think, function, etc. In conditions where there is axon loss and regrowth, there may be recovery of motor and sensory abilities. As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

Among the conditions of interest for the present methods of enhancing synaptogenesis are senescence, stroke, spinal cord injury, Alzheimer's disease (a disease where synapses are lost), as well as promoting new synaptogenesis in repair and regeneration of injured CNS after stroke or spinal cord injury. Such conditions benefit from administration of a Tenm agonist, which increases, or enhances, the development of synapses. In some instances, where there has been neuronal loss, it may be desirable to enhance neurogenesis as well, e.g. through administration of agents or regimens that increase neurogenesis, transplantation of neuronal progenitors, etc.

The term ‘synaptopathy’ refers to brain disorders that have arisen from synaptic dysfunction. Increasing evidence demonstrates the importance of synapse dysfunction as a major determinant of several neurodevelopmental diseases including autism spectrum disorders (ASD), Down syndrome, startle disease, and epilepsy; and neurodegenerative diseases, e.g. Alzheimer and Parkinson disease.

Autism spectrum disorders (ASD) form a group of diverse neurodevelopmental conditions defined by two core symptoms: social deficits that include communication and interaction impairments; and stereotypical, repetitive, and restricted behaviors. ASD is a very complex group of disorders associated with aberrant synaptic transmission and plasticity.

Down syndrome (DS) is a genetic form of learning disability and occurs in ~10 of 10 000 live births. Individuals with DS show deficits in learning and memory, language and executive functions since early childhood, as a result of the presence of an extra copy of chromosome 21 (Hsa21). DS patients exhibit an age-dependent reduction in dendritic branching and spine density that likely involves impaired reorganization of the actin cytoskeleton in neurons, and show early onset Alzheimer-like neurodegeneration. An over-inhibition of synapses by increased GABAergic circuitry may be the main force for synaptic dysfunctions in DS as it affects the excitation-inhibition balance in the brain, which may alter synaptic connectivity and cognitive function. Approaches targeting altered neurotransmitter systems and synaptic function, including the excitation-inhibition imbalance, have been proposed as therapeutic interventions to ameliorate cognitive deficits in DS.

Alzheimer disease (AD) is a neurodegenerative disorder that accounts for around 60% of all dementia cases. AD is a highly sporadic and idiopathic disease, with only a small fraction of cases (1-5%) clearly attributable to familial mutations. The most common AD-linked mutations are located in the amyloid-β precursor protein (APP) gene and in gene regions encoding the catalytic cores of the gamma-secretases presenilin 1 (PSEN1) or presenilin 2 (PSEN2). For sporadic AD, many genetic susceptibility factors have been described to date, the most important being apolipoprotein E (APOE) allele 4.

Soluble Aβ oligomers, instead of deposited plaques, are now thought to underlie widespread neurotoxicity and synaptic loss and the degree of dementia in patients correlates well with the levels of oligomers in post mortem brains. Levels of Aβ oligomers are increased in the brains of AD patients over control brains, and their synaptic presence is linked to poorer cognition regardless of classical neuropathology. Studies in transgenic animal models of AD have shown that synaptic and cognitive impairments are associated with the elevation in soluble oligomeric Aβ species prior to the appearance of plaques and tangles.

The precise mechanisms of Aβ toxicity are not fully understood, but it is believed that oligomers can interact with a number of synaptic proteins including mGluRs and iGluRs, α7-nicotinic acetylcholine receptors, p75 neurotrophin receptor, cellular prion protein, and PSD95. The synaptic action of Aβ triggers aberrant activation of NMDARs, Ca²⁺ deregulation, and cellular stress, which may lead to synaptic dysfunction and neuronal loss. Soluble Aβ further exacerbates neurotransmitter release at excitatory synapses, including glutamate and the NMDAR co-agonist D-serine, thereby triggering post-synaptic neurotoxicity and synapse failure. The fact that cognitive decline appears in patients before extensive neuronal loss is detected reinforces the notion that synapse dysfunction is likely to be an early cause for memory loss in AD. Tau pathology appears to be itself a substantial contributor of synapse dysfunction in AD, and has been recently proposed to propagate across brain structures, in a manner similar to AB. As expected for a complex disease, AB, tau, and APOE4 features appear to interact to promote cognitive decline and neurodegeneration in AD, whereas they may independently contribute to synapse malfunction and cognitive impairment. APOE4 is known to stimulate brain Aβ deposition and, in turn, Aβ promotes abnormal tau phosphorylation to mediate synapse and memory defects in mouse models of AD. APOE4 appears to impair synapses, at least in part, through the action of Aβ oligomers and tau. Thus, multiple factors could act as initiators of common signaling pathways that ultimately lead to synapse impairment and AD neuropathology. The notion that several upstream pathogenic mechanisms act synergistically to promote synapse dysfunction/loss indicate that strategies aimed at preventing synapse failure could provide greater therapeutic benefit for cognitive decline in AD.

Parkinson disease (PD) is characterized by progressive degeneration of nigrostriatal dopaminergic neurons, leading to loss of motor function, rigidity, postural instability, tremor, and bradykinesia. Both familial (5-10% of all cases) and acquired (90-95% of all cases) forms of Parkinsonism are usually caused by defects in dopamine (DA) metabolism. PD leads to profound immobility and disability that usually affects people over the age of 60. Decreases in DA inputs from basal ganglia result in impaired control of motor circuits that ultimately leads to clinical symptoms. Deposition of Lewy bodies (LB), protein aggregates-containing alpha-synuclein (α-syn) often found in remaining dopaminergic neurons, is regarded as central to the pathogenic mechanisms underlying PD. Notably, synapse dysfunction contributes to the progression of PD, with impairments in protein trafficking, autophagy, and mitochondrial function.

Dopaminergic neurons within the substantia nigra (SN) preferentially degenerate within PD pathogenesis. Synaptic dystrophy characterizes the early stages of PD neuropathology. SNARE protein complexes, which mediate the membrane fusion of exocytosis vesicles, are prone to misfolding and non-specific interactions, thus depending on efficient chaperones to remove them from the synapse. α-syn is one such oligomerization-prone chaperones that cause mislocalization of SNARE complexes associated with decreased DA release and synaptic dystrophy. It is thus possible that dysfunctional dopaminergic synapses contribute to disease progression even before evident neurodegeneration takes place.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). These focal ischemia models are in contrast to global ischemia models where blood flow to the entire brain is blocked for a period of time prior to reperfusion. Certain regions of the brain are particularly sensitive to this type of ischemic insult. The precise region of the brain that is directly affected is dictated by the location of the blockage and duration of ischemia prior to reperfusion. One model for focal cerebral ischemia uses middle cerebral artery occlusion (MCAO) in rats. Studies in normotensive rats can produce a standardized and repeatable infarction. MCAO in the rat mimics the increase in plasma catecholamines, electrocardiogramas, sympathetic nerve discharge, and myocytolysis seen in the human patient population.

The methods of the invention are also useful for treatment of injuries to the central nervous system that are caused by mechanical forces, such as a blow to the head or spine, and which, in the absence of treatment, result in neuronal death, or severing of axons. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

Senescence refers to the effects or the characteristics of increasing age, particularly with respect to the diminished ability of somatic tissues to regenerate in response to damage, disease, and normal use. Alternatively, aging may be defined in terms of general physiological characteristics. The rate of aging is very species specific, where a human may be aged at about 50 years; and a rodent at about 2 years. In general terms, a natural progressive decline in body systems starts in early adulthood, but it becomes most evident several decades later. One arbitrary way to define old age more precisely in humans is to say that it begins at conventional retirement age, around about 60, around about 65 years of age. Another definition sets parameters for aging coincident with the loss of reproductive ability, which is around about age 45, more usually around about 50 in humans, but will, however, vary with the individual. Loss of synaptic function may be found in aged individuals.

The essential features of dementia are impairment of short-term memory and long-term memory, abstract thinking, and judgment; other disturbances of higher cortical function; and personality change. Progression of cognitive impairment confirms the diagnosis, and patients with Alzheimer's disease do not improve.

The methods of the invention find also find use in combination with cell or tissue transplantation to the central nervous system, where such grafts include neural progenitors such as those found in fetal tissues, neural stem cells, embryonic stem cells or other cells and tissues contemplated for neural repair or augmentation. Neural stem/progenitor cells have been described in the art, and their use in a variety of therapeutic protocols has been widely discussed. For example, inter alia, U.S. Pat. No. 6,638,501, Bjornson et al.; U.S. Pat. No. 6,541,255, Snyder et al.; U.S. Pat. No. 6,498,018, Carpenter; U.S. patent application No. 20020012903, Goldman et al.; Palmer et al. (2001) Nature 411 (6833):42-3; Palmer et al. (1997) Mol Cell Neurosci. 8(6):389-404; Svendsen et al. (1997) Exp. Neurol. 148(1):135-46 and Shihabuddin (1999) Mol Med Today. 5(11):474-80; each herein specifically incorporated by reference.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. A such, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like. By providing synaptogenesis enhancers, the functional connections of the neurons are enhanced, providing for an improved clinical outcome.

### Methods of Treatment

Enhancing synaptogenesis through administering compounds that are agonists of Tenm is used to promote an improved outcome from ischemic cerebral injury, or other neuronal injury, by modulating synaptogenesis and cellular changes that promote functional improvement. The methods are also used to enhance synaptogenesis in patients suffering from neurodegenerative disorders, e.g. Alzheimer's disease, epilepsy, etc.

Patients can suffer neurological and functional deficits after stroke, CNS injury, and neurodegenerative disease. The findings of the present invention provide a means to enhance synapse formation and to improve function after CNS damage or degeneration. The induction of neural connections induced by promoting synaptogenesis will promote functional improvement after stroke, injury, aging and neurodegenerative disease. The amount of increased synaptogenesis after treatment may comprise at least a measurable increase relative to a control lacking such treatment, for example at least a 10% increase, at least a 20% increase, at least a 50% increase, or more.

The Tenm agonists are administered at a dosage that enhances synaptogenesis while minimizing any side-effects. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

The therapeutic dose may be at least about 0.01 μg/kg body weight, at least about 0.05 ug/kg body weight; at least about 0.1 μg/kg body weight, at least about 0.5 μg/kg body weight, at least about 1 μg/kg body weight, at least about 2.5 μg/kg body weight, at least about 5 μg/kg body weight, at least 10 μg/kg body weight, at least 100 μg/kg body weight, at least 500 μg/kg body weight, at least 1 mg/kg, and not more than about 100 mg/kg, not more than about 50 mg/kg, not more than about 10 mg/kg. It will be understood by one of skill in the art that such guidelines will be adjusted for the molecular weight of the active agent. The dosage may also be varied for localized administration, e.g. intranasal, inhalation, etc., or for systemic administration, e.g. i.m., i.p., i.v., and the like.

The effective amount of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials.

Therapeutic agents, e.g. agonists or analogs can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, e.g. intravenous, intraperitoneal, intradermal, transdermal, sub-cutaneous, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere. Liquid dosage forms for oral administration can contain coloring and flavoring to increase patient acceptance.

Formulations suitable for parenteral or intracranial administration include aqueous and non-aqueous, isotonic sterile injection solutions, which can contain antioxidants, buffers, bacteriostats, and solutes that render the formulation isotonic with the blood or cerebrospinal fluid of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain. Intrathecal administration maybe carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In some embodiments of the invention, a kit is provided, comprising a therapeutic composition for modulation of synaptogenesis, and instructions for use. The therapeutic composition, e.g. a Tenm agonist thereof as described herein, may be provided in a unit dose suitable for administration to an individual.

### EXPERIMENTAL

### Teneurin is a Receptor for SPARCL1

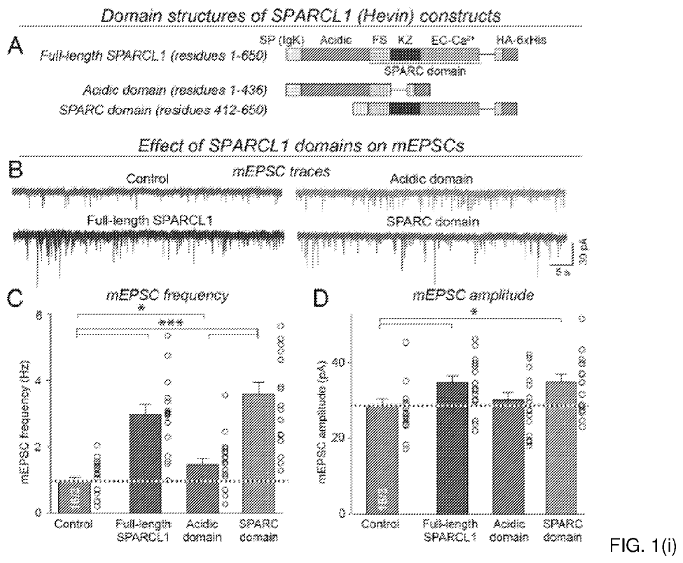

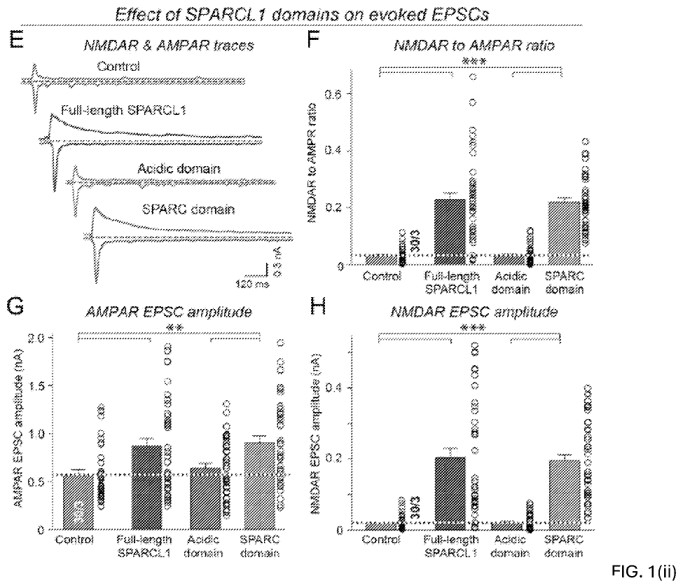

As shown in FIG. 1, electrophysiological recordings from human neurons treated with full-length SPARCL1 and SPARCL1 fragments identifies the SPARCL1-C domain as the critical synaptogenic region of SPARCL1. Full-length SPARCL1 and its C-terminal SPARCL1-C domain but not its N-terminal acidic region potently promote synapse formation as monitored by measurements of spontaneous miniature mEPSCs. This finding is further confirmed by measurements of evoked EPSCs, which show that full-length SPARCL1 and its C-terminal SPARCL1-C domain but not its N-terminal acidic region enhance synaptic connectivity in cultured human neurons, and that they enhance the NMDAR-mediated responses much more strongly than AMPAR-mediated responses.

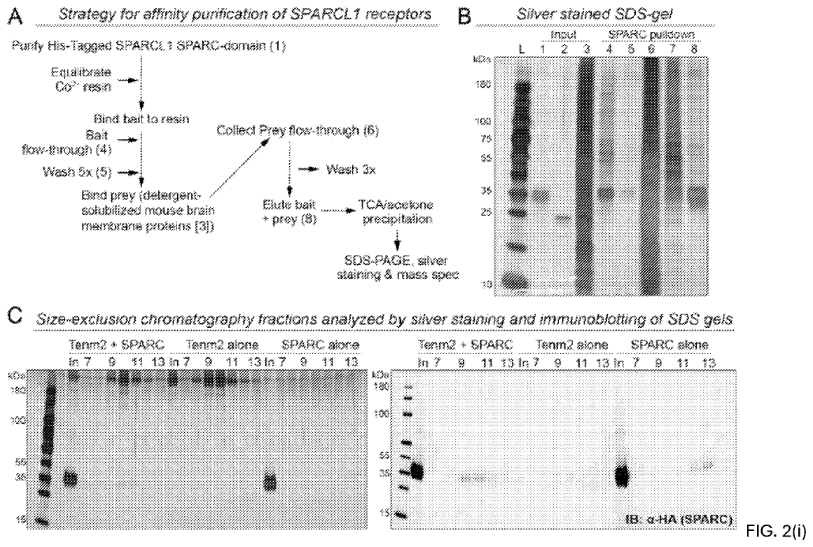

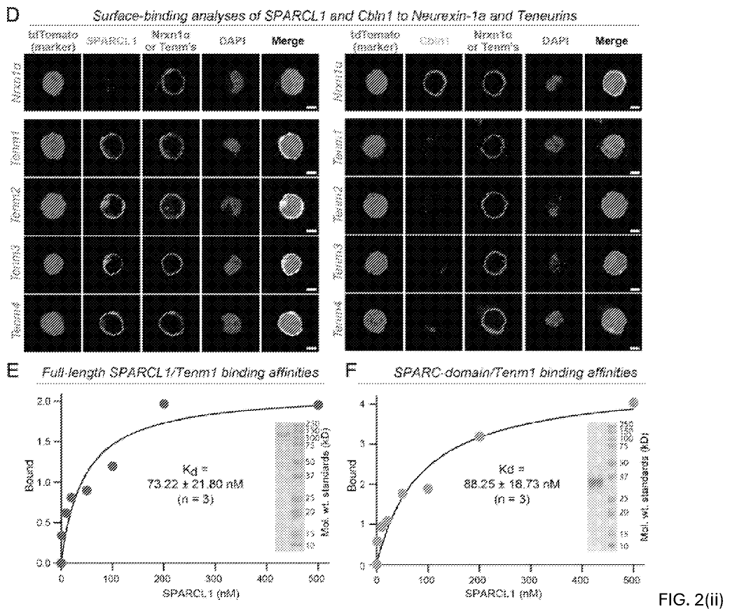

To determine the receptor for SPARCL1, shown in FIG. 2, the synaptogenic domain (SPARCL1-C) was his-tagged, purified, and bound to a resin. Detergent solubilized brain protein extracts were bound to the resin, washed, and eluted. The eluate was examined by gel electrophoresis and mass spectroscopy, where teneurin was identified as the binding partner. To confirm binding of SPARCL1 to teneurins, recombinant proteins composed of either the extracellular domains of Tenm2 alone or of SPARCL1 alone or of both mixed together were analyzed by size-exclusion chromatography. SPARCL1 was only detectable in higher-molecular weight fractions when Tenm2 is present, but not when it is absent. This was confirmed by surface binding of SPARCL1 or Cbln1 (a neurexin ligand used as a control) to HEK293 cells expressing Neurexin-1α (Nrxn1a) or Tenm1-4. SPARCL1 binds to all teneurins but not to neurexin-1α, whereas Cbln1 exhibits the reverse binding pattern. Affinity determinations of the interaction of Tenm1 with full-length SPARCL1 (E) or the C-terminal SPARCL1-C domain (F), demonstrated a high-affinity interaction.

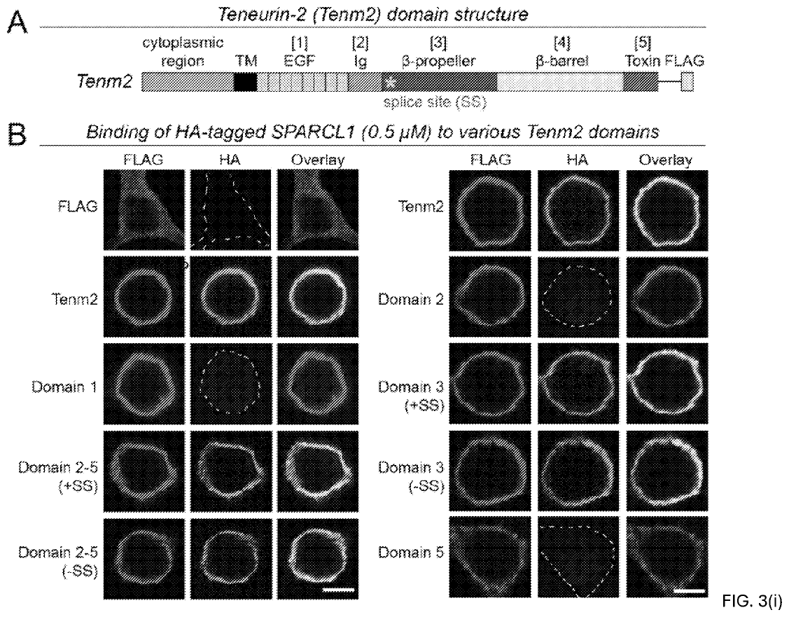

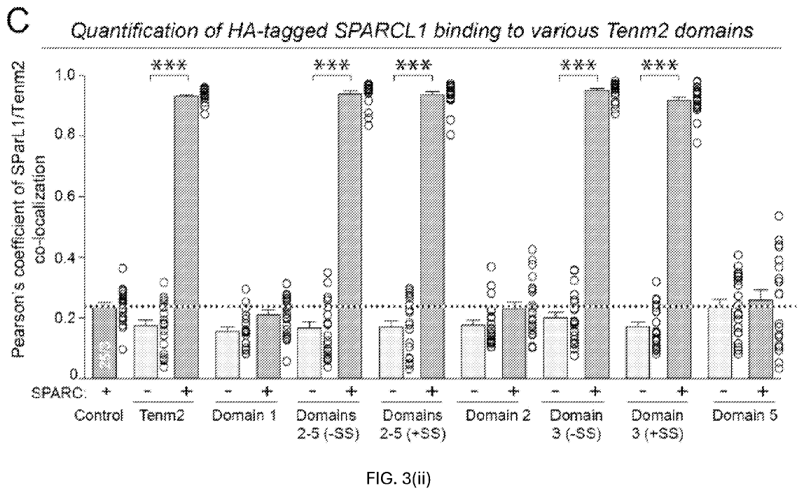

Domain 3 of teneurin-2 was identified as the SPARCL1-CL1 binding site, shown FIGS. 3A-3C. The domain structure of teneurin-2 (Tenm2) is shown in FIG. 3A, where the β-propellor region corresponds to domain 3 (provided herein as SEQ ID NO:2). FIG. 3B shows representative images of SPARCL1 binding to HEK293 cells expressing the indicated Tenm2 domain constructs, consisting of the listed domains fused to the transmembrane region (FLAG=control). It can be seen that only constructs comprising domain 3 bind HA-tagged SPARCL1. Similarly, quantitation of binding is shown in FIG. 3C, where strong binding is shown to domain 3 in the absence or presence of the splice site sequence.

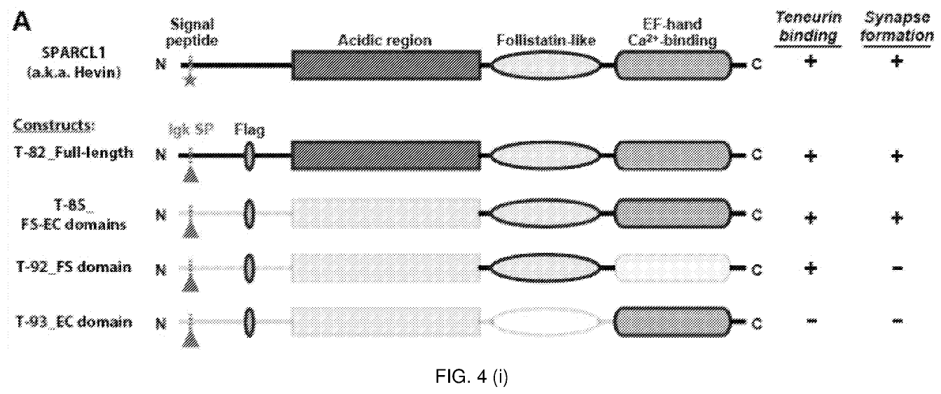

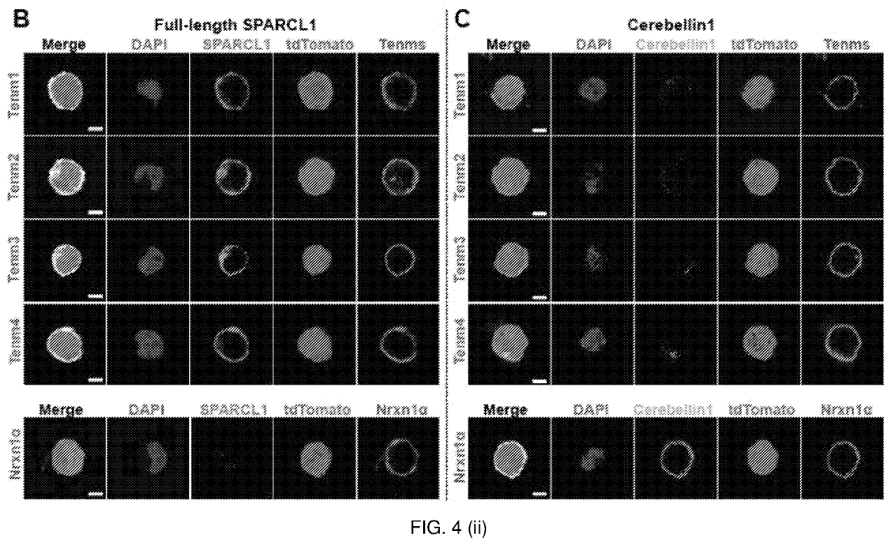

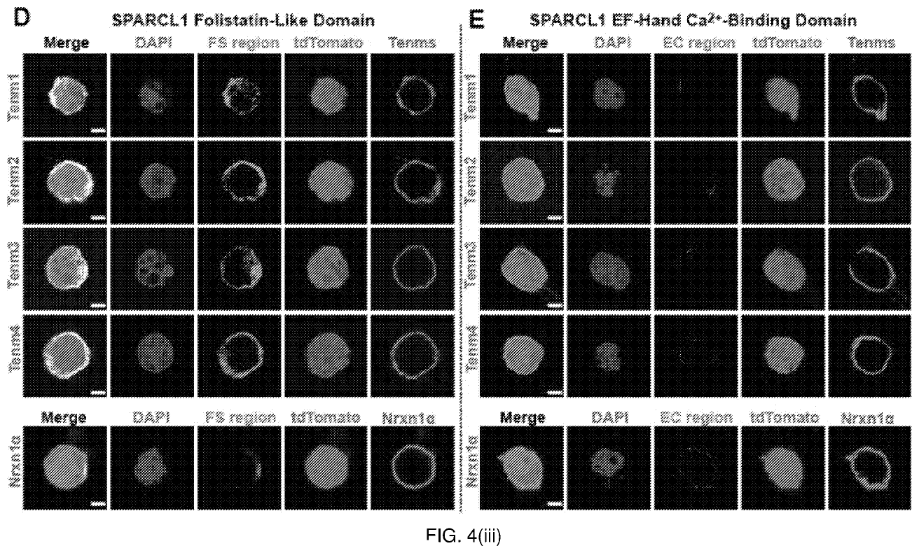

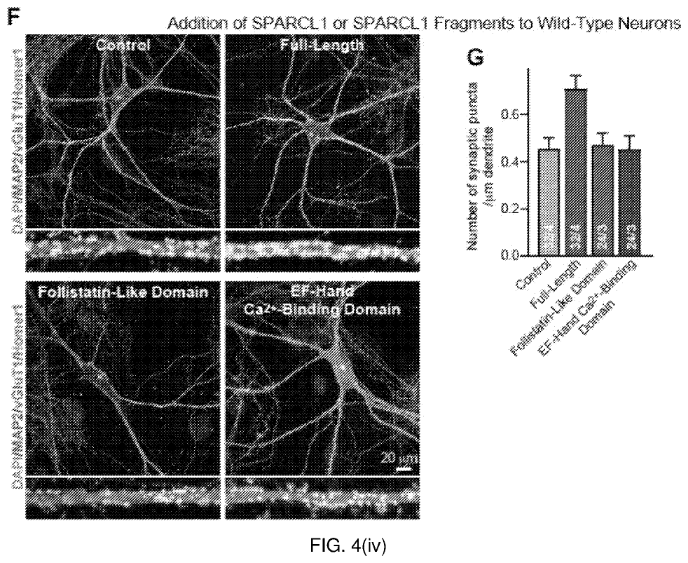

The follistatin-like domain of SPARCL1 is shown to be sufficient for teneurin binding but is unable to boost synapse numbers in cultured neurons, as shown in FIG. 4. Domain structures of SPARCL1 constructs are shown in FIG. 4A. With reference to the Pubmed refseq NP_004675, herein specifically incorporated by reference, the follistatin-like domain comprises residues 433-512, and the EF-hand Ca⁺⁺ binding domain comprises residues 515-655. The entire human SPARC domain (see FIG. 1A), is provided herein as SEQ ID NO:6. The FS and EC domains (SEQ ID NO:6) are sufficient for teneurin binding and synapse formation. The mouse SPARC domain is provided herein as SEQ ID NO:7, residues 384-684.

FIGS. 4B-E illustrate binding assays. Full-length SPARCL1 binds to each of Tenm 1-4, (FIG. 4B). The negative control, Cerebelllin1, (FIG. 4C) does not bind the teneurins. The EF-hand domain does not, by itself, bind teneurins (FIG. 4E), but the Follistatin-like domain (FIG. 4D) does bind. Shown in FIG. 4F-G, the follistatin-like domain of SPARCL1 that binds to teneurins is by itself unable to promote synapse formation different from full-length SPARCL1 (F, representative images; G, quantification of synapse density).

### Sequences

The amino acid sequence corresponding to the domain 3 (NHL repeats/β-propeller) of human Tenm1-4 proteins can be as follows:

human Tenm1 (SEQ ID NO:1):

NGHQRSVACTNCNGPAHNNKLFAPVALASGPDGSVYVGDFNFVRRIFPSGNSVSILELRNRD
TRHSTSPAHKYYLAMDPVSESLYLSDTNTRKVYKLKSLVETKDLSKNFEVVAGTGDQCLPFDQ
SHCGDGGRASEASLNSPRGITVDRHGFIYFVDGTMIRKIDENAVITTVIGSNGLTSTQPLSCDS
GMDITQVRLEWPTDLAVNPMDNSLYVLDNNIVLQISENRRVRIIAGRPIHCQVPGIDHFLVSKVA
IHSTLESARAISVSHSGLLFIAETDERKVNRIQQVTTNGEIYIIAGAPTDCDCKIDPNCDCFSGDG
GYAKDAKMKAPSSLAVSPDGTLYVADLGNVRIRTISRNQAHLNDMNIYEIASPADQEL
Tenm2 (SEQ ID NO: 2):
GNKLLAPVALAVGIDGSLYVGDFNYIRRIFPSRNVTSILELRNKEFKHSNNPAHKYYLAVDPVSG
SLYVSDTNSRRIYRVKSLSGTKDLAGNSEVVAGTGEQCLPFDEARCGDGGKAIDATLMSPRGI
AVDKNGLMYFVDATMIRKVDQNGIISTLLGSNDLTAVRPLSCDSSMDVAQVRLEWPTDLAVNP
MDNSLYVLENNVILRITENHQVSIIAGRPMHCQVPGIDYSLSKLAIHSALESASAIAISHTGVLYIT
ETDEKKINRLRQVTTNGEICLLAGAASDCDCKNDVNCNCYSGDDAYATDAILNSPSSLAVAPD
GTIYIADLGNIRIRAVSKNKPVLNAF
Tenm3 (SEQ ID NO: 3):
CGIDGSLYVGDFNYVRRIFPSGNVTSVLELSSNPAHRYYLATDPVTGDLYVSDTNTRRIYRPKS
LTGAKDLTKNAEVVAGTGEQCLPFDEARCGDGGKAVEATLMSPKGMAVDKNGLIYFVDGTMI
RKVDQNGIISTLLGSNDLTSARPLTCDTSMHISQVRLEWPTDLAINPMDNSIYVLDNNVVLQITE
NRQVRIAAGRPMHCQVPGVEYPVGKHAVQTTLESATAIAVSYSGVLYITETDEKKINRIRQVTT
DGEISLVAGIPSECDCKNDANCDCYQSGDGYAKDAKLSAPSSLAASPDGTLYIADLGNIRIRAV
SKNKPLLNSMNFYEVASPTDQEL
Tenm4 (SEQ ID NO: 4):
VSQQPPVIGSIMGNGRRRSISCPSCNGLADGNKLLAPVALTCGSDGSLYVGDFNYIRRIFPSGN
VTNILELRNKDFRHSHSPAHKYYLATDPMSGAVFLSDSNSRRVFKIKSTVVVKDLVKNSEVVAG
TGDQCLPFDDTRCGDGGKATEATLTNPRGITVDKFGLIYFVDGTMIRRIDQNGIISTLLGSNDLT
SARPLSCDSVMDISQVHLEWPTDLAINPMDNSLYVLDNNVVLQISENHQVRIVAGRPMHCQVP
GIDHFLLSKVAIHATLESATALAVSHNGVLYIAETDEKKINRIRQVTTSGEISLVAGAPSGCDCKN
DANCDCFSGDDGYAKDAKLNTPSSLAVCADGELYVADLGNIRIRFIRKNKPFLNTQNMYELSS
PIDQEL

The amino acid sequence of human full-length SPARCL1 (including a signal sequence of residues 1-16) is as follows (SEQ ID NO:5):

MKTGLFFLCLLGTAAAIPTNARLLSDHSKPTAETVAPDNTAIPSLRAEAEENEKETAVSTEDDSH
HKAEKSSVLKSKEESHEQSAEQGKSSSQELGLKDQEDSDGHLSVNLEYAPTEGTLDIKEDMS
EPQEKKLSENTDFLAPGVSSFTDSNQQESITKREENQEQPRNYSHHQLNRSSKHSQGLRDQG
NQEQDPNISNGEEEEEKEPGEVGTHNDNQERKTELPREHANSKQEEDNTQSDDILEESDQPT
QVSKMQEDEFDQGNQEQEDNSNAEMEEENASNVNKHIQETEWQSQEGKTGLEAISNHKETE
EKTVSEALLMEPTDDGNTTPRNHGVDDDGDDDGDDGGTDGPRHSASDDYFIPSQAFLEAER
AQSIAYHLKIEEQREKVHENENIGTTEPGEHQEAKKAENSSNEEETSSEGNMRVHAVDSCMSF
QCKRGHICKADQQGKPHCVCQDPVTCPPTKPLDQVCGTDNQTYASSCHLFATKCRLEGTKK
GHQLQLDYFGACKSIPTCTDFEVIQFPLRMRDWLKNILMQLYEANSEHAGYLNEKQRNKVKKI
YLDEKRLLAGDHPIDLLLRDFKKNYHMYVYPVHWQFSELDQHPMDRVLTHSELAPLRASLVPM
EHCITRFFEECDPNKDKHITLKEWGHCFGIKEEDIDENLLF

The amino acid sequence of the two C-terminal domains (FSH & SPARCL1-C domains) of human SPARCL1, which represent residues 412-650 of the mature protein are as follows (SEQ ID NO:6):

NSSNEEETSSEGNMRVHAVDSCMSFQCKRGHICKADQQGKPHCVCQDPVTCPPTKPLDQVC
GTDNQTYASSCHLFATKCRLEGTKKGHQLQLDYFGACKSIPTCTDFEVIQFPLRMRDWLKNIL
MQLYEANSEHAGYLNEKQRNKVKKIYLDEKRLLAGDHPIDLLLRDFKKNYHMYVYPVHWQFSE
LDQHPMDRVLTHSELAPLRASLVPMEHCITRFFEECDPNKDKHITLKEWGHC

The amino acid sequence of mature mouse SPARCL1 (SEQ ID NO:7) is:

IPTSTRFLSDHSNPTTATLVTPEDATVPIAGVEATADIENHPNDKAEKPSALNSEEETHEQSTEQ
DKTYSFEVDLKDEEDGDGDLSVDPTEGTLTLDLQEGTSEPQQKSLPENGDFPATVSTSYVDP
NQRANITKGKESQEQPVSDSHQQPNESSKQTQDLKAEESQTQDPDIPNEEEEEEEDEEEEEE
EEPEDIGAPSDNQEEGKEPLEEQPTSKWEGNREQSDDTLEESSQPTQISKTEKHQSEQGNQ
GQESDSEAEGEDKAAGSKEHIPHTEQQDQEGKAGLEAIGNQKDTDEKAVSTEPTDAAVVPRS
HGGAGDNGGGDDSKHGAGDDYFIPSQEFLEAERMHSLSYYLKYGGGEETTTGESENRREAA
DNQEAKKAESSPNAEPSDEGNSREHSAGSCTNFQCKRGHICKTDPQGKPHCVCQDPETCPP
AKILDQACGTDNQTYASSCHLFATKCRLEGTKKGHQLQLDYFGACKSIPACTDFEVAQFPLRM
RDWLKNILMQLYEPNPKHGGYLNEKQRSKVKKIYLDEKRLLAGDHPIELLLRDFKKNYHMYVY
PVHWQFNELDQHPADRILTHSELAPLRASLVPMEHCITRFFEECDPNKDKHITLKEWGHCFGIK
EEDIDENLLF

The amino acid sequence of the mouse N terminal SPARCL1 sequence (corresponding to SEQ ID NO:7, residues 384-684) is:

PNAEPSDEGNSREHSAGSCTNFQCKRGHICKTDPQGKPHCVCQDPETCPPAKILDQACGTD
NQTYASSCHLFATKCRLEGTKKGHQLQLDYFGACKSIPACTDFEVAQFPLRMRDWLKNILMQL
YEPNPKHGGYLNEKQRSKVKKIYLDEKRLLAGDHPIELLLRDFKKNYHMYVYPVHWQFNELDQ
HPADRILTHSELAPLRASLVPMEHCITRFFEECDPNKDKHITLKEWGHCFGIKEEDIDENLLF

The amino acid sequence of mouse SPARCL1 FS domain (corresponding to SEQ ID NO:7, residues 384-483) is

PNAEPSDEGNSREHSAGSCTNFQCKRGHICKTDPQGKPHCVCQDPETCPPAKILDQACGTD
NQTYASSCHLFATKCRLEGTKKGHQLQLDYFGACKSIP

The amino acid sequence of mouse SPARCL1 EC domain (corresponding to SEQ ID NO:7, residues 483-684) is

ACTDFEVAQFPLRMRDWLKNILMQLYEPNPKHGGYLNEKQRSKVKKIYLDEKRLLAGDHPIELL
LRDFKKNYHMYVYPVHWQFNELDQHPADRILTHSELAPLRASLVPMEHCITRFFEECDPNKDK
HITLKEWGHCFGIKEEDIDENLLF

The complete amino acid sequence of a mature human Tenm1 construct (starting with methionine) (SEQ ID NO:8) is:

MEQTDCKPYQPLPKVKHEMDLAYTSSSDESEDGRKPRQSYNSRETLHEYNQELRMNYNSQS
RKRKEVEKSTQEMEFCETSHTLCSGYQTDMHSVSRHGYQLEMGSDVDTETEGAASPDHALR
MWIRGMKSEHSSCLSSRANSALSLTDTDHERKSDGENGFKFSPVCCDMEAQAGSTQDVQSS
PHNQFTFRPLPPPPPPPHACTCARKPPPAADSLQRRSMTTRSQPSPAAPAPPTSTQDSVHLH
NSWVLNSNIPLETRHFLFKHGSGSSAIFSAASQNYPLTSNTVYSPPPRPLPRSTFSRPAFTFNK
PYRCCNWKCTALSATAITVTLALLLAYVIAVHLFGLTWQLQPVEGELYANGVSKGNRGTESMD
TTYSPIGGKVSDKSEKKVFQKGRAIDTGEVDIGAQVMQTIPPGLFWRFQITIHHPIYLKFNISLAK
DSLLGIYGRRNIPPTHTQFDFVKLMDGKQLVKQDSKGSDDTQHSPRNLILTSLQETGFIEYMDQ
GPWYLAFYNDGKKMEQVFVLTTAIEIMDDCSTNCNGNGECISGHCHCFPGFLGPDCARDSCP
VLCGGNGEYEKGHCVCRHGWKGPECDVPEEQCIDPTCFGHGTCIMGVCICVPGYKGEICEEE
DCLDPMCSNHGICVKGECHCSTGWGGVNCETPLPVCQEQCSGHGTFLLDAGVCSCDPKWT
GSDCSTELCTMECGSHGVCSRGICQCEEGWVGPTCEERSCHSHCTEHGQCKDGKCECSPG
WEGDHCTIAHYLDAVRDGCPGLCFGNGRCTLDQNGWHCVCQVGWSGTGCNVVMEMLCGD
NLDNDGDGLTDCVDPDCCQQSNCYISPLCQGSPDPLDLIQQSQTLFSQHTSRLFYDRIKFLIGK
DSTHVIPPEVSFDSRRACVIRGQVVAIDGTPLVGVNVSFLHHSDYGFTISRQDGSFDLVAIGGIS
VILIFDRSPFLPEKRTLWLPWNQFIVVEKVTMQRVVSDPPSCDISNFISPNPIVLPSPLTSFGGS
CPERGTIVPELQVVQEEIPIPSSFVRLSYLSSRTPGYKTLLRILLTHSTIPVGMIKVHLTVAVEGRL
TQKWFPAAINLVYTFAWNKTDIYGQKVWGLAEALVSVGYEYETCPDFILWEQRTVVLQGFEM
DASNLGGWSLNKHHILNPQSGIIHKGNGENMFISQQPPVISTIMGNGHQRSVACTNCNGPAHN
NKLFAPVALASGPDGSVYVGDFNFVRRIFPSGNSVSILELRNRDTRHSTSPAHKYYLAMDPVS
ESLYLSDTNTRKVYKLKSLVETKDLSKNFEVVAGTGDQCLPFDQSHCGDGGRASEASLNSPR
GITVDRHGFIYFVDGTMIRKIDENAVITTVIGSNGLTSTQPLSCDSGMDITQVRLEWPTDLAVNP
MDNSLYVLDNNIVLQISENRRVRIIAGRPIHCQVPGIDHFLVSKVAIHSTLESARAISVSHSGLLFI
AETDERKVNRIQQVTTNGEIYIIAGAPTDCDCKIDPNCDCFSGDGGYAKDAKMKAPSSLAVSPD
GTLYVADLGNVRIRTISRNQAHLNDMNIYEIASPADQELYQFTVNGTHLHTLNLITRDYVYNFTY
NSEGDLGAITSSNGNSVHIRRDAGGMPLWLVVPGGQVYWLTISSNGVLKRVSAQGYNLALMT
YPGNTGLLATKSNENGWTTVYEYDPEGHLTNATFPTGEVSSFHSDLEKLTKVELDTSNRENVL
MSTNLTATSTIYILKQENTQSTYRVNPDGSLRVTFASGMEIGLSSEPHILAGAVNPTLGKCNISL
PGEHNANLIEWRQRKEQNKGNVSAFERRLRAHNRNLLSIDFDHITRTGKIYDDHRKFTLRILYD
QTGRPILWSPVSRYNEVNITYSPSGLVTFIQRGTWNEKMEYDQSGKIISRTWADGKIWSYTYLE
KSVMLLLHSQRRYIFEYDQPDCLLSVTMPSMVRHSLQTMLSVGYYRNIYTPPDSSTSFIQDYS
RDGRLLQTLHLGTGRRVLYKYTKQARLSEVLYDTTQVTLTYEESSGVIKTIHLMHDGFICTIRYR
QTGPLIGRQIFRFSEEGLVNARFDYSYNNFRVTSMQAVINETPLPIDLYRYVDVSGRTEQFGKF
SVINYDLNQVITTTVMKHTKIFSANGQVIEVQYEILKAIAYWMTIQYDNVGRMVICDIRVGVDANI
TRYFYEYDADGQLQTVSVNDKTQWRYSYDLNGNINLLSHGKSARLTPLRYDLRDRITRLGEIQ
YKMDEDGFLRQRGNDIFEYNSNGLLQKAYNKASGWTVQYYYDGLGRRVASKSSLGQHLQFF
YADLTNPIRVTHLYNHTSSEITSLYYDLQGHLIAMELSSGEEYYVACDNTGTPLAVFSSRGQVIK
EILYTPYGDIYHDTYPDFQVIIGFHGGLYDFLTKLVHLGQRDYDVVAGRWTTPNHHIWKQLNLL
PKPFNLYSFENNYPVGKIQDVAKYTTDIRSWLELFGFQLHNVLPGFPKPELENLELTYELLRLQ
TKTQEWDPGKTILGIQCELQKQLRNFISLDQLPMTPRYNDGRCLEGGKQPRFAAVPSVFGKGI
KFAIKDGIVTADIIGVANEDSRRLAAILNNAHYLENLHFTIEGRDTHYFIKLGSLEEDLVLIGNTGG
RRILENGVNVTVSQMTSVLNGRTRRFADIQLQHGALCFNIRYGTTVEEEKNHVLEIARQRAVAQ
AWTKEQRRLQEGEEGIRAWTEGEKQQLLSTGRVQGYDGYFVLSVEQYLELSDSANNIHFMR
QSEIGRR

The complete amino acid sequence of a mature human Tenm2 construct starting with methionine (SEQ ID NO:9) is:

MDVKDRRHRSLTRGRCGKECRYTSSSLDSEDCRVPTQKSYSSSETLKAYDHDSRMHYGNRV
TDLIHRESDEFPRQGTNFTLAELGICEPSPHRSGYCSDMGILHQGYSLSTGSDADSDTEGGMS
PEHAIRLWGRGIKSRRSSGLSSRENSALTLTDSDNENKSDDENGRPIPPTSSPSLLPSAQLPSS
HNPPPVSCQMPLLDSNTSHQIMDTNPDEEFSPNSYLLRACSGPQQASSSGPPNHHSQSTLRP
PLPPPHNHTLSHHHSSANSLNRNSLTNRRSQIHAPAPAPNDLATTPESVQLQDSWVLNSNVPL
ETRHFLFKTSSGSTPLFSSSSPGYPLTSGTVYTPPPRLLPRNTFSRKAFKLKKPSKYCSWKCA
ALSAIAAALLLAILLAYFIAMHLLGLNWQLQPADGHTFNNGIRTGLPGNDDVATMPSGGKVPWS
LKNSSIDSGEAEVGRRVTQEVPPGVFWRSQIHISQPQFLKFNISLGKDALFGVYIRRGLPPSHA
QYDFMERLDGKEKWSVVESPRERRSIQTLVQNEAVFVQYLDVGLWHLAFYNDGKDKEMVSF
NTVVLDSVQDCPRNCHGNGECVSGVCHCFPGFLGADCAKAACPVLCSGNGQYSKGTCQCY
SGWKGAECDVPMNQCIDPSCGGHGSCIDGNCVCSAGYKGEHCEEVDCLDPTCSSHGVCVN
GECLCSPGWGGLNCELARVQCPDQCSGHGTYLPDTGLCSCDPNWMGPDCSVEVCSVDCGT
HGVCIGGACRCEEGWTGAACDQRVCHPRCIEHGTCKDGKCECREGWNGEHCTIDGCPDLC
NGNGRCTLGQNSWQCVCQTGWRGPGCNVAMETSCADNKDNEGDGLVDCLDPDCCLQSAC
QNSLLCRGSRDPLDIIQQGQTDWPAVKSFYDRIKLLAGKDSTHIIPGENPFNSSLVSLIRGQVVT
TDGTPLVGVNVSFVKYPKYGYTITRQDGTFDLIANGGASLTLHFERAPFMSQERTVWLPWNSF
YAMDTLVMKTEENSIPSCDLSGFVRPDPIIISSPLSTFFSAAPGQNPIVPETQVLHEEIELPGSNV
KLRYLSSRTAGYKSLLKITMTQSTVPLNLIRVHLMVAVEGHLFQKSFQASPNLAYTFIWDKTDA
YGQRVYGLSDAVVSVGFEYETCPSLILWEKRTALLQGFELDPSNLGGWSLDKHHILNVKSGIL
HKGTGENQFLTQQPAIITSIMGNGRRRSISCPSCNGLAEGNKLLAPVALAVGIDGSLYVGDFNYI
RRIFPSRNVTSILELRNKEFKHSNNPAHKYYLAVDPVSGSLYVSDTNSRRIYRVKSLSGTKDLA
GNSEVVAGTGEQCLPFDEARCGDGGKAIDATLMSPRGIAVDKNGLMYFVDATMIRKVDQNGII
STLLGSNDLTAVRPLSCDSSMDVAQVRLEWPTDLAVNPMDNSLYVLENNVILRITENHQVSIIA
GRPMHCQVPGIDYSLSKLAIHSALESASAIAISHTGVLYITETDEKKINRLRQVTTNGEICLLAGA
ASDCDCKNDVNCNCYSGDDAYATDAILNSPSSLAVAPDGTIYIADLGNIRIRAVSKNKPVLNAF
NQYEAASPGEQELYVFNADGIHQYTVSLVTGEYLYNFTYSTDNDVTELIDNNGNSLKIRRDSSG
MPRHLLMPDNQIITLTVGTNGGLKVVSTQNLELGLMTYDGNTGLLATKSDETGWTTFYDYDHE
GRLTNVTRPTGVVTSLHREMEKSITIDIENSNRDDDVTVITNLSSVEASYTVVQDQVRNSYQLC
NNGTLRVMYANGMGISFHSEPHVLAGTITPTIGRCNISLPMENGLNSIEWRLRKEQIKGKVTIFG
RKLRVHGRNLLSIDYDRSIRTEKIYDDHRKFTLRIIYDQVGRPFLWLPSSGLAAVNVSYFFNGRL
AGLQRGAMSERTDIDKQGRIVSRMFADGKVWSYSYLDKSMVLLLQSQRQYIFEYDSSDRLLA
VTMPSVARHSMSTHTSIGYIRNIYNPPESNASVIFDYSDDGRILKTSFLGTGRQVFYKYGKLSKL
SEIVYDSTAVTFGYDETTGVLKMVNLQSGGFSCTIRYRKIGPLVDKQIYRFSEEGMVNARFDYT
YHDNSFRIASIKPVISETPLPVDLYRYDEISGKVEHFGKFGVIYYDINQIITTAVMTLSKHFDTHGR
IKEVQYEMFRSLMYWMTVQYDSMGRVIKRELKLGPYANTTKYTYDYDGDGQLQSVAVNDRPT
WRYSYDLNGNLHLLNPGNSVRLMPLRYDLRDRITRLGDVQYKIDDDGYLCQRGSDIFEYNSKG
LLTRAYNKASGWSVQYRYDGVGRRASYKTNLGHHLQYFYSDLHNPTRITHVYNHSNSEITSLY
YDLQGHLFAMESSSGEEYYVASDNTGTPLAVFSINGLMIKQLQYTAYGEIYYDSNPDFQMVIGF
HGGLYDPLTKLVHFTQRDYDVLAGRWTSPDYTMWKNVGKEPAPFNLYMFKSNNPLSSELDLK
NYVTDVKSWLVMFGFQLSNIIPGFPRAKMYFVPPPYELSESQASENGQLITGVQQTTERHNQA
FMALEGQVITKKLHASIREKAGHWFATTTPIIGKGIMFAIKEGRVTTGVSSIASEDSRKVASVLNN
AYYLDKMHYSIEGKDTHYFVKIGSADGDLVTLGTTIGRKVLESGVNVTVSQPTLLVNGRTRRFT
NIEFQYSTLLLSIRYGLTPDTLDEEKARVLDQARQRALGTAWAKEQQKARDGREGSRLWTEG
EKQQLLSTGRVQGYEGYYVLPVEQYPELADSSSNIQFLRQNEMGKR

The complete amino acid sequence of a mature mouse Tenm3 construct starting with methionine (SEQ ID NO:10) is:

MDVKERRPYCSLTKSRREKERRYTNSSADNEECRVPTQKSYSSSETLKAFDHDSSRLLYGNR
VKDLVHREADEYTRQGQNFTLRQLGVCESATRRGVAFCAEMGLPHRGYSISAGSDADTENEA
VMSPEHAMRLWGRGVKSGRSSCLSSRSNSALTLTDTEHENRSDSESEQPSNNPGQPTLQPL
PPSHKQHPAQHHPSITSLNRNSLTNRRNQSPAPPAALPAELQTTPESVQLQDSWVLGSNVPL
ESRHFLFKTGTGTTPLFSTATPGYTMASGSVYSPPTRPLPRNTLSRSAFKFKKSSKYCSWRCT
ALCAVGVSVLLAILLSYFIAMHLFGLNWHLQQTENDTFENGKVNSDTVPTNTVSLPSGDNGKL
GGFTHENNTIDSGELDIGRRAIQEVPPGIFWRSQLFIDQPQFLKFNISLQKDALIGVYGRKGLPP
SHTQYDFVELLDGSRLIAREQRNLVESERAGRQARSVSLHEAGFIQYLDSGIWHLAFYNDGKN
PEQVSFNTIVIESVVECPRNCHGNGECVSGTCHCFPGFLGPDCSRAACPVLCSGNGQYSKGR
CLCFSGWKGTECDVPTTQCIDPQCGGRGICIMGSCACNSGYKGENCEEADCLDPGCSNHGV
CIHGECHCNPGWGGSNCEILKTMCADQCSGHGTYLQESGSCTCDPNWTGPDCSNEICSVDC
GSHGVCMGGSCRCEEGWTGPACNQRACHPRCAEHGTCKDGKCECSQGWNGEHCTIEGCP
GLCNSNGRCTLDQNGWHCVCQPGWRGAGCDVAMETLCTDSKDNEGDGLIDCMDPDCCLQ
SSCQNQPYCRGLPDPQDIISQSLQTPSQQAAKSFYDRISFLIGSDSTHVLPGESPFNKSLASVI
RGQVLTADGTPLIGVNVSFLHYSEYGYTITRQDGMFDLVANGGASLTLVFERSPFLTQYHTVWI
PWNVFYVMDTLVMKKEENDIPSCDLSGFVRPSPIIVSSPLSTFFRSSPEDSPIIPETQVLHEETTI
PGTDLKLSYLSSRAAGYKSVLKITMTQAVIPFNLMKVHLMVAVVGRLFQKWFPASPNLAYTFIW
DKTDAYNQKVYGLSEAVVSVGYEYESCLDLTLWEKRTAVLQGYELDASNMGGWTLDKHHVL
DVQNGILYKGNGENQFISQQPPVVSSIMGNGRRRSISCPSCNGQADGNKLLAPVALACGIDGS
LYVGDFNYVRRIFPSGNVTSVLELSSNPAHRYYLATDPVTGDLYVSDTNTRRIYRPKSLTGAKD
LTKNAEVVAGTGEQCLPFDEARCGDGGKAVEATLMSPKGMAIDKNGLIYFVDGTMIRKVDQN
GIISTLLGSNDLTSARPLTCDTSMHISQVRLEWPTDLAINPMDNSIYVLDNNVVLQITENRQVRIA
AGRPMHCQVPGVEYPVGKHAVQTTLESATAIAVSYSGVLYITETDEKKINRIRQVTTDGEISLVA
GIPSECDCKNDANCDCYQSGDGYAKDAKLNAPSSLAASPDGTLYIADLGNIRIRAVSKNKPLLN
SMNFYEVASPTDQELYIFDINGTHQYTVSLVTGDYLYNFSYSNDNDVTAVTDSNGNTLRIRRDP
NRMPVRVVSPDNQVIWLTIGTNGCLKSMTAQGLELVLFTYHGNSGLLATKSDETGWTTFFDYD
SEGRLTNVTFPTGVVTNLHGDMDKAITVDIESSSREEDVSITSNLSSIDSFYTMVQDQLRNSYQI
GYDGSLRIFYASGLDSHYQTEPHVLAGTANPTVAKRNMTLPGENGQNLVEWRFRKEQAQGK
VNVFGRKLRVNGRNLLSVDFDRTTKTEKIYDDHRKFLLRIAYDTSGHPTLWLPSSKLMAVNVTY
SSTGQIASIQRGTTSEKVDYDSQGRIVSRVFADGKTWSYTYLEKSMVLLLHSQRQYIFEYDMW
DRLSAITMPSVARHTMQTIRSIGYYRNIYNPPESNASIITDYNEEGLLLQTAFLGTSRRVLFKYRR
QTRLSEILYDSTRVSFTYDETAGVLKTVNLQSDGFICTIRYRQIGPLIDRQIFRFSEDGMVNARF
DYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKI
MWRYNYDLNGNLHLLNPSSSARLTPLRYDLRDRITRLGDVQYRLDEDGFLRQRGTEIFEYSSK
GLLTRVYSKGSGWTVIYRYDGLGRRVSSKTSLGQHLQFFYADLTYPTRITHVYNHSSSEITSLY
YDLQGHLFAMEISSGDEFYIASDNTGTPLAVFSSNGLMLKQIQYTAYGEIYFDSNVDFQLVIGFH
GGLYDPLTKLIHFGERDYDILAGRWTTPDIEIWKRIGKDPAPFNLYMFRNNNPASKIHDVKDYIT
DVNSWLVTFGFHLHNAIPGFPVPKFDLTEPSYELVKSQQWEDVPPIFGVQQQVARQAKAFLSL
GKMAEVQVSRRKAGAEQSWLWFATVKSLIGKGVMLAVSQGRVQTNVLNIANEDCIKVAAVLN
NAFYLENLHFTIEGKDTHYFIKTTTPESDLGTLRLTSGRKALENGINVTVSQSTTVVNGRTRRFA
DVEMQFGALALHVRYGMTLDEEKARILEQARQRALARAWAREQQRVRDGEEGARLWTEGEK
RQLLSAGKVQGYDGYYVLSVEQYPELADSANNIQFLRQSEIGKR

The complete amino acid sequence of a mature mouse Tenm4 construct starting with methionine (SEQ ID NO:11) is:

MEPDHSALSAARAQFVDVEEREPEAMDVKERKPYRSLTRRRDAERRYTSSSADSEEGKGPQ
KSYSSSETLKAYDQDARLAYGSRVKDMVPQEAEEFCRTGTNFTLRELGLGEMTPPHGTLYRT
DIGLPHCGYSMGASSDADLEADTVLSPEHPVRLWGRSTRSGRSSCLSSRANSNLTLTDTEHE
NTETDHPSSLQNHPRLRTPPPPLPHAHTPNQHHAASINSLNRGNFTPRSNPSPAPTDHSLSGE
PPAGSAQEPTHAQDNWLLNSNIPLETRNLGKQPFLGTLQDNLIEMDILSASRHDGAYSDGHFL
FKPGGTSPLFCTTSPGYPLTSSTVYSPPPRPLPRSTFSRPAFNLKKPSKYCNWKCAALSAILIS
ATLVILLAYFVAMHLFGLNWHLQPMEGQMQMYEITEDTASSWPVPTDVSLYPSGGTGLETPD
RKGKGAAEGKPSSLFPEDSFIDSGEIDVGRRASQKIPPGTFWRSQVFIDHPVHLKFNVSLGKA
ALVGIYGRKGLPPSHTQFDFVELLDGRRLLTQEARSLEGPQRQSRGPVPPSSHETGFIQYLDS
GIWHLAFYNDGKESEVVSFLTTAIESVDNCPSNCYGNGDCISGTCHCFLGFLGPDCGRASCPV
LCSGNGQYMKGRCLCHSGWKGAECDVPTNQCIDVACSSHGTCIMGTCICNPGYKGESCEEV
DCMDPTCSSRGVCVRGECHCSVGWGGTNCETPRATCLDQCSGHGTFLPDTGLCNCDPSWT
GHDCSIEICAADCGGHGVCVGGTCRCEDGWMGAACDQRACHPRCAEHGTCRDGKCECSPG
WNGEHCTIAHYLDRVVKEGCPGLCNGNGRCTLDLNGWHCVCQLGWRGTGCDTSMETGCGD
GKDNDGDGLVDCMDPDCCLQPLCHVNPLCLGSPDPLDIIQETQAPVSQQNLNSFYDRIKFLVG
RDSTHSIPGENPFDGGHACVIRGQVMTSDGTPLVGVNISFINNPLFGYTISRQDGSFDLVTNGG
ISIILRFERAPFITQEHTLWLPWDRFFVMETIVMRHEENEIPSCDLSNFARPNPVVSPSPLTSFAS
SCAEKGPIVPEIQALQEEIVIAGCKMRLSYLSSRTPGYKSVLRISLTHPTIPFNLMKVHLMVAVEG
RLFRKWFAAAPDLSYYFIWDKTDVYNQKVFGLSEAFVSVGYEYESCPDLILWEKRTAVLQGYE
IDASKLGGWSLDKHHALNIQSGILHKGNGENQFVSQQPPVIGSIMGNGRRRSISCPSCNGLAD
GNKLLAPVALTCGSDGSLYVGDFNYIRRIFPSGNVTNILEMRNKDFRHSHSPAHKYYLATDPM
SGAVFLSDTNSRRVFKVKSTTVVKDLVKNSEVVAGTGDQCLPFDDTRCGDGGKATEATLTNP
RGITVDKFGLIYFVDGTMIRRVDQNGIISTLLGSNDLTSARPLSCDSVMEISQVRLEWPTDLAIN
PMDNSLYVLDNNVVLQISENHQVRIVAGRPMHCQVPGIDHFLLSKVAIHATLESATALAVSHNG
VLYIAETDEKKINRIRQVTTSGEISLVAGAPSGCDCKNDANCDCFSGDDGYAKDAKLNTPSSLA
VCADGELYVADLGNIRIRFIRKNKPFLNTQNMYELSSPIDQELYLFDTSGKHLYTQSLPTGDYLY
NFTYTGDGDITHITDNNGNMVNVRRDSTGMPLWLVVPDGQVYWVTMGTNSALRSVTTQGHE
LAMMTYHGNSGLLATKSNENGWTTFYEYDSFGRLTNVTFPTGQVSSFRSDTDSSVHVQVETS
SKDDVTITTNLSASGAFYTLLQDQVRNSYYIGADGSLRLLLANGMEVALQTEPHLLAGTVNPTV
GKRNVTLPIDNGLNLVEWRQRKEQARGQVTVFGRRLRVHNRNLLSLDFDRVTRTEKIYDDHR
KFTLRILYDQAGRPSLWSPSSRLNGVNVTYSPGGHIAGIQRGIMSERMEYDQAGRITSRIFADG
KMWSYTYLEKSMVLHLHSQRQYIFEFDKNDRLSSVTMPNVARQTLETIRSVGYYRNIYQPPEG
NASVIQDFTEDGHLLHTFYLGTGRRVIYKYGKLSKLAETLYDTTKVSFTYDETAGMLKTVNLQN
EGFTCTIRYRQIGPLIDRQIFRFTEEGMVNARFDYNYDNSFRVTSMQAVINETPLPIDLYRYDDV
SGKTEQFGKFGVIYYDINQIITTAVMTHTKHFDAYGRMKEVQYEIFRSLMYWMTVQYDNMGRV
VKKELKVGPYANTTRYSYEYDADGQLQTVSINDKPLWRYSYDLNGNLHLLSPGNSARLTPLRY
DLRDRITRLGDVQYKMDEDGFLRQRGGDVFEYNSAGLLIKAYNRASGWSVRYRYDGLGRRV
SSKSSHSHHLQFFYADLTNPTKVTHLYNHSSSEITSLYYDLQGHLFAMELSSGDEFYIACDNIG
TPLAVFSGTGLMIKQILYTAYGEIYMDTNPNFQIIIGYHGGLYDPLTKLVHMGRRDYDVLAGRWT
SPDHELWKRLSSNSIVPFHLYMFKNNNPISNSQDIKCFMTDVNSWLLTFGFQLHNVIPGYPKPD
TDAMEPSYELVHTQMKTQEWDNSKSILGVQCEVQKQLKAFVTLERFDQLYGSTITSCQQAPE
TKKFASSGSIFGKGVKFALKDGRVTTDIISVANEDGRRIAAILNNAHYLENLHFTIDGVDTHYFVK
PGPSEGDLAILGLSGGRRTLENGVNVTVSQINTVLSGRTRRYTDIQLQYRALCLNTRYGTTVDE
EKVRVLELARQRAVRQAWAREQQRLREGEEGLRAWTDGEKQQVLNTGRVQGYDGFFVTSV
EQYPELSDSANNIHFMRQSEMGRR

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of enhancing synaptogenesis, comprising the step of:
contacting a neuron with an effective dose of a teneurin (Tenm) agonist; wherein the number of synapses is increased in the neuron relative to a neuron that is not contacted with a teneurin agonist.

2. The method of claim 1, wherein the Tenm agonist binds to domain 3 of teneurin.

3. The method of claim 1, wherein the teneurin is selected from human TENM1, TENM2, TENM3 and TENM4.

4. The method of claim 1, wherein the teneurin is human TENM2.

5. The method of claim 1, wherein the agonist comprises all or a portion of a SPARCL1 protein SPARC domain.

6. The method of claim 5, wherein the SPARC domain comprises or consists of SEQ ID NO:6 or SEQ ID NO:7, residues 384-684.

7. The method of claim 5, wherein the agonist comprises an analog of SEQ ID NO:6 or SEQ ID NO:7, residues 384-684.

8. The method of claim 1, wherein the Tenm agonist is a polypeptide comprising an antigen binding region (ABR).

9. The method of claim 8, wherein the polypeptide comprising an antigen binding region is an antibody or binding fragment derived therefrom.

10. The method of claim 7, wherein the polypeptide comprising an antigen binding region is a VHH protein.

11. The method of claim 1, wherein the agonist is a small molecule drug.

12. The method of claim 1, wherein the contacting step is performed in vitro.

13. The method of claim 1, wherein the Tenm agonist is administered to an individual suffering from adverse effects of deficits in synapse function.

14. The method of claim 13, wherein the individual has synapse loss associated with Alzheimer's disease, Parkinson's disease; traumatic brain injury, or epilepsy.

15. A method of screening a candidate agent for activity as a Tenm agonist, the method comprising:
contacting domain 3 of a teneurin protein with a candidate agent to determine if the candidate agent specifically binds to the domain 3 of the teneurin protein, wherein a candidate Tenm agonist specifically binds to the domain 3 of a teneurin protein.

16. The method of claim 15, further comprising a step of determining if the candidate agent activates teneurin signaling, wherein an agent that activates teneurin signaling is a candidate synaptogenic agent.

17. The method of claim 15, wherein the teneurin is selected from human TENM1, TENM2, TENM3 and TENM4.

18. The method of claim 17, wherein the teneurin is human TENM2.

19. The method of claim 15, wherein the candidate agent comprises a small molecule library.

20. The method of claim 15, wherein the candidate agent comprises an antibody library.


SEQUENCE LISTING (11 sequences)

SEQ ID NO: 1 - protein, 378 aa, Homo sapiens
NGHQRSVACTNCNGPAHNNKLFAPVALASGPDGSVYVGDFNFVRRIFPSGNSVSILELRNRDTRHSTSPAHKYYLAMDPVSESLYLSDTNTRKVYKLKSLVETKDLSKNFEVVAGTGDQCLPFDQSHCGDGGRASEASLNSPRGITVDRHGFIYFVDGTMIRKIDENAVITTVIGSNGLTSTQPLSCDSGMDITQVRLEWPTDLAVNPMDNSLYVLDNNIVLQISENRRVRIIAGRPIHCQVPGIDHFLVSKVAIHSTLESARAISVSHSGLLFIAETDERKVNRIQQVTTNGEIYIIAGAPTDCDCKIDPNCDCFSGDGGYAKDAKMKAPSSLAVSPDGTLYVADLGNVRIRTISRNQAHLNDMNIYEIASPADQEL

SEQ ID NO: 2 - protein, 347 aa, Homo sapiens
GNKLLAPVALAVGIDGSLYVGDFNYIRRIFPSRNVTSILELRNKEFKHSNNPAHKYYLAVDPVSGSLYVSDTNSRRIYRVKSLSGTKDLAGNSEVVAGTGEQCLPFDEARCGDGGKAIDATLMSPRGIAVDKNGLMYFVDATMIRKVDQNGIISTLLGSNDLTAVRPLSCDSSMDVAQVRLEWPTDLAVNPMDNSLYVLENNVILRITENHQVSIIAGRPMHCQVPGIDYSLSKLAIHSALESASAIAISHTGVLYITETDEKKINRLRQVTTNGEICLLAGAASDCDCKNDVNCNCYSGDDAYATDAILNSPSSLAVAPDGTIYIADLGNIRIRAVSKNKPVLNAF

SEQ ID NO: 3 - protein, 342 aa, Homo sapiens
CGIDGSLYVGDFNYVRRIFPSGNVTSVLELSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLSAPSSLAASPDGTLYIADLGNIRIRAVSKNKPLLNSMNFYEVASPTDQEL

SEQ ID NO: 4 - protein, 391 aa, Homo sapiens
VSQQPPVIGSIMGNGRRRSISCPSCNGLADGNKLLAPVALTCGSDGSLYVGDFNYIRRIFPSGNVTNILELRNKDFRHSHSPAHKYYLATDPMSGAVFLSDSNSRRVFKIKSTVVVKDLVKNSEVVAGTGDQCLPFDDTRCGDGGKATEATLTNPRGITVDKFGLIYFVDGTMIRRIDQNGIISTLLGSNDLTSARPLSCDSVMDISQVHLEWPTDLAINPMDNSLYVLDNNVVLQISENHQVRIVAGRPMHCQVPGIDHFLLSKVAIHATLESATALAVSHNGVLYIAETDEKKINRIRQVTTSGEISLVAGAPSGCDCKNDANCDCFSGDDGYAKDAKLNTPSSLAVCADGELYVADLGNIRIRFIRKNKPFLNTQNMYELSSPIDQEL

SEQ ID NO: 5 - protein, 664 aa, Homo sapiens
MKTGLFFLCLLGTAAAIPTNARLLSDHSKPTAETVAPDNTAIPSLRAEAEENEKETAVSTEDDSHHKAEKSSVLKSKEESHEQSAEQGKSSSQELGLKDQEDSDGHLSVNLEYAPTEGTLDIKEDMSEPQEKKLSENTDFLAPGVSSFTDSNQQESITKREENQEQPRNYSHHQLNRSSKHSQGLRDQGNQEQDPNISNGEEEEEKEPGEVGTHNDNQERKTELPREHANSKQEEDNTQSDDILEESDQPTQVSKMQEDEFDQGNQEQEDNSNAEMEEENASNVNKHIQETEWQSQEGKTGLEAISNHKETEEKTVSEALLMEPTDDGNTTPRNHGVDDDGDDDGDDGGTDGPRHSASDDYFIPSQAFLEAERAQSIAYHLKIEEQREKVHENENIGTTEPGEHQEAKKAENSSNEEETSSEGNMRVHAVDSCMSFQCKRGHICKADQQGKPHCVCQDPVTCPPTKPLDQVCGTDNQTYASSCHLFATKCRLEGTKKGHQLQLDYFGACKSIPTCTDFEVIQFPLRMRDWLKNILMQLYEANSEHAGYLNEKQRNKVKKIYLDEKRLLAGDHPIDLLLRDFKKNYHMYVYPVHWQFSELDQHPMDRVLTHSELAPLRASLVPMEHCITRFFEECDPNKDKHITLKEWGHCFGIKEEDIDENLLF

SEQ ID NO: 6 - protein, 239 aa, Homo sapiens
NSSNEEETSSEGNMRVHAVDSCMSFQCKRGHICKADQQGKPHCVCQDPVTCPPTKPLDQVCGTDNQTYASSCHLFATKCRLEGTKKGHQLQLDYFGACKSIPTCTDFEVIQFPLRMRDWLKNILMQLYEANSEHAGYLNEKQRNKVKKIYLDEKRLLAGDHPIDLLLRDFKKNYHMYVYPVHWQFSELDQHPMDRVLTHSELAPLRASLVPMEHCITRFFEECDPNKDKHITLKEWGHC

SEQ ID NO: 7 - protein, 634 aa, Mus musculus
IPTSTRFLSDHSNPTTATLVTPEDATVPIAGVEATADIENHPNDKAEKPSALNSEEETHEQSTEQDKTYSFEVDLKDEEDGDGDLSVDPTEGTLTLDLQEGTSEPQQKSLPENGDFPATVSTSYVDPNQRANITKGKESQEQPVSDSHQQPNESSKQTQDLKAEESQTQDPDIPNEEEEEEEDEEEEEEEEPEDIGAPSDNQEEGKEPLEEQPTSKWEGNREQSDDTLEESSQPTQISKTEKHQSEQGNQGQESDSEAEGEDKAAGSKEHIPHTEQQDQEGKAGLEAIGNQKDTDEKAVSTEPTDAAVVPRSHGGAGDNGGGDDSKHGAGDDYFIPSQEFLEAERMHSLSYYLKYGGGEETTTGESENRREAADNQEAKKAESSPNAEPSDEGNSREHSAGSCTNFQCKRGHICKTDPQGKPHCVCQDPETCPPAKILDQACGTDNQTYASSCHLFATKCRLEGTKKGHQLQLDYFGACKSIPACTDFEVAQFPLRMRDWLKNILMQLYEPNPKHGGYLNEKQRSKVKKIYLDEKRLLAGDHPIELLLRDFKKNYHMYVYPVHWQFNELDQHPADRILTHSELAPLRASLVPMEHCITRFFEECDPNKDKHITLKEWGHCFGIKEEDIDENLLF

SEQ ID NO: 8 - protein, 2732 aa, Homo sapiens
MEQTDCKPYQPLPKVKHEMDLAYTSSSDESEDGRKPRQSYNSRETLHEYNQELRMNYNSQSRKRKEVEKSTQEMEFCETSHTLCSGYQTDMHSVSRHGYQLEMGSDVDTETEGAASPDHALRMWIRGMKSEHSSCLSSRANSALSLTDTDHERKSDGENGFKFSPVCCDMEAQAGSTQDVQSSPHNQFTFRPLPPPPPPPHACTCARKPPPAADSLQRRSMTTRSQPSPAAPAPPTSTQDSVHLHNSWVLNSNIPLETRHFLFKHGSGSSAIFSAASQNYPLTSNTVYSPPPRPLPRSTFSRPAFTFNKPYRCCNWKCTALSATAITVTLALLLAYVIAVHLFGLTWQLQPVEGELYANGVSKGNRGTESMDTTYSPIGGKVSDKSEKKVFQKGRAIDTGEVDIGAQVMQTIPPGLFWRFQITIHHPIYLKFNISLAKDSLLGIYGRRNIPPTHTQFDFVKLMDGKQLVKQDSKGSDDTQHSPRNLILTSLQETGFIEYMDQGPWYLAFYNDGKKMEQVFVLTTAIEIMDDCSTNCNGNGECISGHCHCFPGFLGPDCARDSCPVLCGGNGEYEKGHCVCRHGWKGPECDVPEEQCIDPTCFGHGTCIMGVCICVPGYKGEICEEEDCLDPMCSNHGICVKGECHCSTGWGGVNCETPLPVCQEQCSGHGTFLLDAGVCSCDPKWTGSDCSTELCTMECGSHGVCSRGICQCEEGWVGPTCEERSCHSHCTEHGQCKDGKCECSPGWEGDHCTIAHYLDAVRDGCPGLCFGNGRCTLDQNGWHCVCQVGWSGTGCNVVMEMLCGDNLDNDGDGLTDCVDPDCCQQSNCYISPLCQGSPDPLDLIQQSQTLFSQHTSRLFYDRIKFLIGKDSTHVIPPEVSFDSRRACVIRGQVVAIDGTPLVGVNVSFLHHSDYGFTISRQDGSFDLVAIGGISVILIFDRSPFLPEKRTLWLPWNQFIVVEKVTMQRVVSDPPSCDISNFISPNPIVLPSPLTSFGGSCPERGTIVPELQVVQEEIPIPSSFVRLSYLSSRTPGYKTLLRILLTHSTIPVGMIKVHLTVAVEGRLTQKWFPAAINLVYTFAWNKTDIYGQKVWGLAEALVSVGYEYETCPDFILWEQRTVVLQGFEMDASNLGGWSLNKHHILNPQSGIIHKGNGENMFISQQPPVISTIMGNGHQRSVACTNCNGPAHNNKLFAPVALASGPDGSVYVGDFNFVRRIFPSGNSVSILELRNRDTRHSTSPAHKYYLAMDPVSESLYLSDTNTRKVYKLKSLVETKDLSKNFEVVAGTGDQCLPFDQSHCGDGGRASEASLNSPRGITVDRHGFIYFVDGTMIRKIDENAVITTVIGSNGLTSTQPLSCDSGMDITQVRLEWPTDLAVNPMDNSLYVLDNNIVLQISENRRVRIIAGRPIHCQVPGIDHFLVSKVAIHSTLESARAISVSHSGLLFIAETDERKVNRIQQVTTNGEIYIIAGAPTDCDCKIDPNCDCFSGDGGYAKDAKMKAPSSLAVSPDGTLYVADLGNVRIRTISRNQAHLNDMNIYEIASPADQELYQFTVNGTHLHTLNLITRDYVYNFTYNSEGDLGAITSSNGNSVHIRRDAGGMPLWLVVPGGQVYWLTISSNGVLKRVSAQGYNLALMTYPGNTGLLATKSNENGWTTVYEYDPEGHLTNATFPTGEVSSFHSDLEKLTKVELDTSNRENVLMSTNLTATSTIYILKQENTQSTYRVNPDGSLRVTFASGMEIGLSSEPHILAGAVNPTLGKCNISLPGEHNANLIEWRQRKEQNKGNVSAFERRLRAHNRNLLSIDFDHITRTGKIYDDHRKFTLRILYDQTGRPILWSPVSRYNEVNITYSPSGLVTFIQRGTWNEKMEYDQSGKIISRTWADGKIWSYTYLEKSVMLLLHSQRRYIFEYDQPDCLLSVTMPSMVRHSLQTMLSVGYYRNIYTPPDSSTSFIQDYSRDGRLLQTLHLGTGRRVLYKYTKQARLSEVLYDTTQVTLTYEESSGVIKTIHLMHDGFICTIRYRQTGPLIGRQIFRFSEEGLVNARFDYSYNNFRVTSMQAVINETPLPIDLYRYVDVSGRTEQFGKFSVINYDLNQVITTTVMKHTKIFSANGQVIEVQYEILKAIAYWMTIQYDNVGRMVICDIRVGVDANITRYFYEYDADGQLQTVSVNDKTQWRYSYDLNGNINLLSHGKSARLTPLRYDLRDRITRLGEIQYKMDEDGFLRQRGNDIFEYNSNGLLQKAYNKASGWTVQYYYDGLGRRVASKSSLGQHLQFFYADLTNPIRVTHLYNHTSSEITSLYYDLQGHLIAMELSSGEEYYVACDNTGTPLAVFSSRGQVIKEILYTPYGDIYHDTYPDFQVIIGFHGGLYDFLTKLVHLGQRDYDVVAGRWTTPNHHIWKQLNLLPKPFNLYSFENNYPVGKIQDVAKYTTDIRSWLELFGFQLHNVLPGFPKPELENLELTYELLRLQTKTQEWDPGKTILGIQCELQKQLRNFISLDQLPMTPRYNDGRCLEGGKQPRFAAVPSVFGKGIKFAIKDGIVTADIIGVANEDSRRLAAILNNAHYLENLHFTIEGRDTHYFIKLGSLEEDLVLIGNTGGRRILENGVNVTVSQMTSVLNGRTRRFADIQLQHGALCFNIRYGTTVEEEKNHVLEIARQRAVAQAWTKEQRRLQEGEEGIRAWTEGEKQQLLSTGRVQGYDGYFVLSVEQYLELSDSANNIHFMRQSEIGRR

SEQ ID NO: 9 - protein, 2765 aa, Homo sapiens
MDVKDRRHRSLTRGRCGKECRYTSSSLDSEDCRVPTQKSYSSSETLKAYDHDSRMHYGNRVTDLIHRESDEFPRQGTNFTLAELGICEPSPHRSGYCSDMGILHQGYSLSTGSDADSDTEGGMSPEHAIRLWGRGIKSRRSSGLSSRENSALTLTDSDNENKSDDENGRPIPPTSSPSLLPSAQLPSSHNPPPVSCQMPLLDSNTSHQIMDTNPDEEFSPNSYLLRACSGPQQASSSGPPNHHSQSTLRPPLPPPHNHTLSHHHSSANSLNRNSLTNRRSQIHAPAPAPNDLATTPESVQLQDSWVLNSNVPLETRHFLFKTSSGSTPLFSSSSPGYPLTSGTVYTPPPRLLPRNTFSRKAFKLKKPSKYCSWKCAALSAIAAALLLAILLAYFIAMHLLGLNWQLQPADGHTFNNGIRTGLPGNDDVATMPSGGKVPWSLKNSSIDSGEAEVGRRVTQEVPPGVFWRSQIHISQPQFLKFNISLGKDALFGVYIRRGLPPSHAQYDFMERLDGKEKWSVVESPRERRSIQTLVQNEAVFVQYLDVGLWHLAFYNDGKDKEMVSFNTVVLDSVQDCPRNCHGNGECVSGVCHCFPGFLGADCAKAACPVLCSGNGQYSKGTCQCYSGWKGAECDVPMNQCIDPSCGGHGSCIDGNCVCSAGYKGEHCEEVDCLDPTCSSHGVCVNGECLCSPGWGGLNCELARVQCPDQCSGHGTYLPDTGLCSCDPNWMGPDCSVEVCSVDCGTHGVCIGGACRCEEGWTGAACDQRVCHPRCIEHGTCKDGKCECREGWNGEHCTIDGCPDLCNGNGRCTLGQNSWQCVCQTGWRGPGCNVAMETSCADNKDNEGDGLVDCLDPDCCLQSACQNSLLCRGSRDPLDIIQQGQTDWPAVKSFYDRIKLLAGKDSTHIIPGENPFNSSLVSLIRGQVVTTDGTPLVGVNVSFVKYPKYGYTITRQDGTFDLIANGGASLTLHFERAPFMSQERTVWLPWNSFYAMDTLVMKTEENSIPSCDLSGFVRPDPIIISSPLSTFFSAAPGQNPIVPETQVLHEEIELPGSNVKLRYLSSRTAGYKSLLKITMTQSTVPLNLIRVHLMVAVEGHLFQKSFQASPNLAYTFIWDKTDAYGQRVYGLSDAVVSVGFEYETCPSLILWEKRTALLQGFELDPSNLGGWSLDKHHILNVKSGILHKGTGENQFLTQQPAIITSIMGNGRRRSISCPSCNGLAEGNKLLAPVALAVGIDGSLYVGDFNYIRRIFPSRNVTSILELRNKEFKHSNNPAHKYYLAVDPVSGSLYVSDTNSRRIYRVKSLSGTKDLAGNSEVVAGTGEQCLPFDEARCGDGGKAIDATLMSPRGIAVDKNGLMYFVDATMIRKVDQNGIISTLLGSNDLTAVRPLSCDSSMDVAQVRLEWPTDLAVNPMDNSLYVLENNVILRITENHQVSIIAGRPMHCQVPGIDYSLSKLAIHSALESASAIAISHTGVLYITETDEKKINRLRQVTTNGEICLLAGAASDCDCKNDVNCNCYSGDDAYATDAILNSPSSLAVAPDGTIYIADLGNIRIRAVSKNKPVLNAFNQYEAASPGEQELYVFNADGIHQYTVSLVTGEYLYNFTYSTDNDVTELIDNNGNSLKIRRDSSGMPRHLLMPDNQIITLTVGTNGGLKVVSTQNLELGLMTYDGNTGLLATKSDETGWTTFYDYDHEGRLTNVTRPTGVVTSLHREMEKSITIDIENSNRDDDVTVITNLSSVEASYTVVQDQVRNSYQLCNNGTLRVMYANGMGISFHSEPHVLAGTITPTIGRCNISLPMENGLNSIEWRLRKEQIKGKVTIFGRKLRVHGRNLLSIDYDRSIRTEKIYDDHRKFTLRIIYDQVGRPFLWLPSSGLAAVNVSYFFNGRLAGLQRGAMSERTDIDKQGRIVSRMFADGKVWSYSYLDKSMVLLLQSQRQYIFEYDSSDRLLAVTMPSVARHSMSTHTSIGYIRNIYNPPESNASVIFDYSDDGRILKTSFLGTGRQVFYKYGKLSKLSEIVYDSTAVTFGYDETTGVLKMVNLQSGGFSCTIRYRKIGPLVDKQIYRFSEEGMVNARFDYTYHDNSFRIASIKPVISETPLPVDLYRYDEISGKVEHFGKFGVIYYDINQIITTAVMTLSKHFDTHGRIKEVQYEMFRSLMYWMTVQYDSMGRVIKRELKLGPYANTTKYTYDYDGDGQLQSVAVNDRPTWRYSYDLNGNLHLLNPGNSVRLMPLRYDLRDRITRLGDVQYKIDDDGYLCQRGSDIFEYNSKGLLTRAYNKASGWSVQYRYDGVGRRASYKTNLGHHLQYFYSDLHNPTRITHVYNHSNSEITSLYYDLQGHLFAMESSSGEEYYVASDNTGTPLAVFSINGLMIKQLQYTAYGEIYYDSNPDFQMVIGFHGGLYDPLTKLVHFTQRDYDVLAGRWTSPDYTMWKNVGKEPAPFNLYMFKSNNPLSSELDLKNYVTDVKSWLVMFGFQLSNIIPGFPRAKMYFVPPPYELSESQASENGQLITGVQQTTERHNQAFMALEGQVITKKLHASIREKAGHWFATTTPIIGKGIMFAIKEGRVTTGVSSIASEDSRKVASVLNNAYYLDKMHYSIEGKDTHYFVKIGSADGDLVTLGTTIGRKVLESGVNVTVSQPTLLVNGRTRRFTNIEFQYSTLLLSIRYGLTPDTLDEEKARVLDQARQRALGTAWAKEQQKARDGREGSRLWTEGEKQQLLSTGRVQGYEGYYVLPVEQYPELADSSSNIQFLRQNEMGKR

SEQ ID NO: 10 - protein, 2699 aa, Mus musculus
MDVKERRPYCSLTKSRREKERRYTNSSADNEECRVPTQKSYSSSETLKAFDHDSSRLLYGNRVKDLVHREADEYTRQGQNFTLRQLGVCESATRRGVAFCAEMGLPHRGYSISAGSDADTENEAVMSPEHAMRLWGRGVKSGRSSCLSSRSNSALTLTDTEHENRSDSESEQPSNNPGQPTLQPLPPSHKQHPAQHHPSITSLNRNSLTNRRNQSPAPPAALPAELQTTPESVQLQDSWVLGSNVPLESRHFLFKTGTGTTPLFSTATPGYTMASGSVYSPPTRPLPRNTLSRSAFKFKKSSKYCSWRCTALCAVGVSVLLAILLSYFIAMHLFGLNWHLQQTENDTFENGKVNSDTVPTNTVSLPSGDNGKLGGFTHENNTIDSGELDIGRRAIQEVPPGIFWRSQLFIDQPQFLKFNISLQKDALIGVYGRKGLPPSHTQYDFVELLDGSRLIAREQRNLVESERAGRQARSVSLHEAGFIQYLDSGIWHLAFYNDGKNPEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACNSGYKGENCEEADCLDPGCSNHGVCIHGECHCNPGWGGSNCEILKTMCADQCSGHGTYLQESGSCTCDPNWTGPDCSNEICSVDCGSHGVCMGGSCRCEEGWTGPACNQRACHPRCAEHGTCKDGKCECSQGWNGEHCTIEGCPGLCNSNGRCTLDQNGWHCVCQPGWRGAGCDVAMETLCTDSKDNEGDGLIDCMDPDCCLQSSCQNQPYCRGLPDPQDIISQSLQTPSQQAAKSFYDRISFLIGSDSTHVLPGESPFNKSLASVIRGQVLTADGTPLIGVNVSFLHYSEYGYTITRQDGMFDLVANGGASLTLVFERSPFLTQYHTVWIPWNVFYVMDTLVMKKEENDIPSCDLSGFVRPSPIIVSSPLSTFFRSSPEDSPIIPETQVLHEETTIPGTDLKLSYLSSRAAGYKSVLKITMTQAVIPFNLMKVHLMVAVVGRLFQKWFPASPNLAYTFIWDKTDAYNQKVYGLSEAVVSVGYEYESCLDLTLWEKRTAVLQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELSSNPAHRYYLATDPVTGDLYVSDTNTRRIYRPKSLTGAKDLTKNAEVVAGTGEQCLPFDEARCGDGGKAVEATLMSPKGMAIDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLNAPSSLAASPDGTLYIADLGNIRIRAVSKNKPLLNSMNFYEVASPTDQELYIFDINGTHQYTVSLVTGDYLYNFSYSNDNDVTAVTDSNGNTLRIRRDPNRMPVRVVSPDNQVIWLTIGTNGCLKSMTAQGLELVLFTYHGNSGLLATKSDETGWTTFFDYDSEGRLTNVTFPTGVVTNLHGDMDKAITVDIESSSREEDVSITSNLSSIDSFYTMVQDQLRNSYQIGYDGSLRIFYASGLDSHYQTEPHVLAGTANPTVAKRNMTLPGENGQNLVEWRFRKEQAQGKVNVFGRKLRVNGRNLLSVDFDRTTKTEKIYDDHRKFLLRIAYDTSGHPTLWLPSSKLMAVNVTYSSTGQIASIQRGTTSEKVDYDSQGRIVSRVFADGKTWSYTYLEKSMVLLLHSQRQYIFEYDMWDRLSAITMPSVARHTMQTIRSIGYYRNIYNPPESNASIITDYNEEGLLLQTAFLGTSRRVLFKYRRQTRLSEILYDSTRVSFTYDETAGVLKTVNLQSDGFICTIRYRQIGPLIDRQIFRFSEDGMVNARFDYSYDNSFRVTSMQGVINETPLPIDLYQFDDISGKVEQFGKFGVIYYDINQIISTAVMTYTKHFDAHGRIKEIQYEIFRSLMYWITIQYDNMGRVTKREIKIGPFANTTKYAYEYDVDGQLQTVYLNEKIMWRYNYDLNGNLHLLNPSSSARLTPLRYDLRDRITRLGDVQYRLDEDGFLRQRGTEIFEYSSKGLLTRVYSKGSGWTVIYRYDGLGRRVSSKTSLGQHLQFFYADLTYPTRITHVYNHSSSEITSLYYDLQGHLFAMEISSGDEFYIASDNTGTPLAVFSSNGLMLKQIQYTAYGEIYFDSNVDFQLVIGFHGGLYDPLTKLIHFGERDYDILAGRWTTPDIEIWKRIGKDPAPFNLYMFRNNNPASKIHDVKDYITDVNSWLVTFGFHLHNAIPGFPVPKFDLTEPSYELVKSQQWEDVPPIFGVQQQVARQAKAFLSLGKMAEVQVSRRKAGAEQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGKR

SEQ ID NO: 11 - protein, 2796 aa, Mus musculus
MEPDHSALSAARAQFVDVEEREPEAMDVKERKPYRSLTRRRDAERRYTSSSADSEEGKGPQKSYSSSETLKAYDQDARLAYGSRVKDMVPQEAEEFCRTGTNFTLRELGLGEMTPPHGTLYRTDIGLPHCGYSMGASSDADLEADTVLSPEHPVRLWGRSTRSGRSSCLSSRANSNLTLTDTEHENTETDHPSSLQNHPRLRTPPPPLPHAHTPNQHHAASINSLNRGNFTPRSNPSPAPTDHSLSGEPPAGSAQEPTHAQDNWLLNSNIPLETRNLGKQPFLGTLQDNLIEMDILSASRHDGAYSDGHFLFKPGGTSPLFCTTSPGYPLTSSTVYSPPPRPLPRSTFSRPAFNLKKPSKYCNWKCAALSAILISATLVILLAYFVAMHLFGLNWHLQPMEGQMQMYEITEDTASSWPVPTDVSLYPSGGTGLETPDRKGKGAAEGKPSSLFPEDSFIDSGEIDVGRRASQKIPPGTFWRSQVFIDHPVHLKFNVSLGKAALVGIYGRKGLPPSHTQFDFVELLDGRRLLTQEARSLEGPQRQSRGPVPPSSHETGFIQYLDSGIWHLAFYNDGKESEVVSFLTTAIESVDNCPSNCYGNGDCISGTCHCFLGFLGPDCGRASCPVLCSGNGQYMKGRCLCHSGWKGAECDVPTNQCIDVACSSHGTCIMGTCICNPGYKGESCEEVDCMDPTCSSRGVCVRGECHCSVGWGGTNCETPRATCLDQCSGHGTFLPDTGLCNCDPSWTGHDCSIEICAADCGGHGVCVGGTCRCEDGWMGAACDQRACHPRCAEHGTCRDGKCECSPGWNGEHCTIAHYLDRVVKEGCPGLCNGNGRCTLDLNGWHCVCQLGWRGTGCDTSMETGCGDGKDNDGDGLVDCMDPDCCLQPLCHVNPLCLGSPDPLDIIQETQAPVSQQNLNSFYDRIKFLVGRDSTHSIPGENPFDGGHACVIRGQVMTSDGTPLVGVNISFINNPLFGYTISRQDGSFDLVTNGGISIILRFERAPFITQEHTLWLPWDRFFVMETIVMRHEENEIPSCDLSNFARPNPVVSPSPLTSFASSCAEKGPIVPEIQALQEEIVIAGCKMRLSYLSSRTPGYKSVLRISLTHPTIPFNLMKVHLMVAVEGRLFRKWFAAAPDLSYYFIWDKTDVYNQKVFGLSEAFVSVGYEYESCPDLILWEKRTAVLQGYEIDASKLGGWSLDKHHALNIQSGILHKGNGENQFVSQQPPVIGSIMGNGRRRSISCPSCNGLADGNKLLAPVALTCGSDGSLYVGDFNYIRRIFPSGNVTNILEMRNKDFRHSHSPAHKYYLATDPMSGAVFLSDTNSRRVFKVKSTTVVKDLVKNSEVVAGTGDQCLPFDDTRCGDGGKATEATLTNPRGITVDKFGLIYFVDGTMIRRVDQNGIISTLLGSNDLTSARPLSCDSVMEISQVRLEWPTDLAINPMDNSLYVLDNNVVLQISENHQVRIVAGRPMHCQVPGIDHFLLSKVAIHATLESATALAVSHNGVLYIAETDEKKINRIRQVTTSGEISLVAGAPSGCDCKNDANCDCFSGDDGYAKDAKLNTPSSLAVCADGELYVADLGNIRIRFIRKNKPFLNTQNMYELSSPIDQELYLFDTSGKHLYTQSLPTGDYLYNFTYTGDGDITHITDNNGNMVNVRRDSTGMPLWLVVPDGQVYWVTMGTNSALRSVTTQGHELAMMTYHGNSGLLATKSNENGWTTFYEYDSFGRLTNVTFPTGQVSSFRSDTDSSVHVQVETSSKDDVTITTNLSASGAFYTLLQDQVRNSYYIGADGSLRLLLANGMEVALQTEPHLLAGTVNPTVGKRNVTLPIDNGLNLVEWRQRKEQARGQVTVFGRRLRVHNRNLLSLDFDRVTRTEKIYDDHRKFTLRILYDQAGRPSLWSPSSRLNGVNVTYSPGGHIAGIQRGIMSERMEYDQAGRITSRIFADGKMWSYTYLEKSMVLHLHSQRQYIFEFDKNDRLSSVTMPNVARQTLETIRSVGYYRNIYQPPEGNASVIQDFTEDGHLLHTFYLGTGRRVIYKYGKLSKLAETLYDTTKVSFTYDETAGMLKTVNLQNEGFTCTIRYRQIGPLIDRQIFRFTEEGMVNARFDYNYDNSFRVTSMQAVINETPLPIDLYRYDDVSGKTEQFGKFGVIYYDINQIITTAVMTHTKHFDAYGRMKEVQYEIFRSLMYWMTVQYDNMGRVVKKELKVGPYANTTRYSYEYDADGQLQTVSINDKPLWRYSYDLNGNLHLLSPGNSARLTPLRYDLRDRITRLGDVQYKMDEDGFLRQRGGDVFEYNSAGLLIKAYNRASGWSVRYRYDGLGRRVSSKSSHSHHLQFFYADLTNPTKVTHLYNHSSSEITSLYYDLQGHLFAMELSSGDEFYIACDNIGTPLAVFSGTGLMIKQILYTAYGEIYMDTNPNFQIIIGYHGGLYDPLTKLVHMGRRDYDVLAGRWTSPDHELWKRLSSNSIVPFHLYMFKNNNPISNSQDIKCFMTDVNSWLLTFGFQLHNVIPGYPKPDTDAMEPSYELVHTQMKTQEWDNSKSILGVQCEVQKQLKAFVTLERFDQLYGSTITSCQQAPETKKFASSGSIFGKGVKFALKDGRVTTDIISVANEDGRRIAAILNNAHYLENLHFTIDGVDTHYFVKPGPSEGDLAILGLSGGRRTLENGVNVTVSQINTVLSGRTRRYTDIQLQYRALCLNTRYGTTVDEEKVRVLELARQRAVRQAWAREQQRLREGEEGLRAWTDGEKQQVLNTGRVQGYDGFFVTSVEQYPELSDSANNIHFMRQSEMGRR